(19) United States
(12) Patent Application Publication
(10) Pub. No.: US 2024/0203521 A1
(43) Pub. Date: Jun. 20, 2024

(54) EVALUATION AND IMPROVEMENT OF GENETIC SCREENING TESTS USING RECEIVER OPERATING CHARACTERISTIC CURVES

(71) Applicant: MYRIAD WOMEN'S HEALTH, INC., South San Francisco, CA (US)

(72) Inventors: Kevin R. Haas, Berkeley, CA (US); Kyle A. Beauchamp, San Mateo, CA (US)

(73) Assignee: MYRIAD WOMEN'S HEALTH, INC., South San Francisco, CA (US)

(21) Appl. No.: 18/592,076

(22) Filed: Feb. 29, 2024

Related U.S. Application Data
(63) Continuation of application No. 16/124,037, filed on Sep. 6, 2018.
(60) Provisional application No. 62/573,122, filed on Oct. 16, 2017.
(60) Provisional application No. 62/555,046, filed on Sep. 6, 2017.

(51) Int. Cl. G16B 5/20; G06N 7/01; G06N 20/00; G16B 30/10; G16B 40/30

(52) U.S. Cl. CPC G16B 5/20; G06N 7/01; G06N 20/00; G16B 30/10; G16B 40/30

(57) ABSTRACT

Described herein are methods for evaluating and improving performance of a genetic screening test for determining a fetal chromosomal abnormality in a test chromosome in a fetus by analyzing a maternal sample of a woman carrying the fetus. The method may include generating simulated maternal samples based on a determined relationship between values of statistical significance and abnormality classifications for the test chromosome of a plurality of reference maternal samples. The method may also include determining specificity values and sensitivity values for a range of abnormality classifier values for the test chromosome based on values of statistical significance and abnormality classifications for the plurality of reference maternal samples and the plurality of simulated maternal samples. A receiver operating characteristic (ROC) curve for the genetic screening test may be generated based on the determined specificity values and sensitivity values.

### CROSS-REFERENCE TO RELATED APPLICATIONS

This application is a continuation of U.S. patent application Ser. No. 16/124,037, filed on Sep. 6, 2018, and titled EVALUATION AND IMPROVEMENT OF GENETIC SCREENING TESTS USING RECEIVER OPERATING CHARACTERISTIC CURVES, which claims the benefit of U.S. Provisional Application No. 62/555,046, filed Sep. 6, 2017, and titled VALIDATION AND OPTIMIZATION OF NONINVASIVE PRENATAL SCREENING and of U.S. Provisional Application No. 62/573, 122, filed Oct. 16, 2017 and titled EVALUATION AND IMPROVEMENT OF GENETIC SCREENING TESTS USING RECEIVER OPERATING CHARACTERISTIC CURVES, the disclosure of each of which is incorporated by reference herein in its entirety for all purposes.

### FIELD OF THE INVENTION

The present invention relates to the genetic screening tests for determining fetal abnormalities in one or more chromosomes from cell-free deoxyribonucleic acid (DNA).

### BACKGROUND

Circulating throughout the bloodstream of a pregnant woman and separate from cellular tissue are small pieces of DNA, often referred to as cell-free DNA (cfDNA). The cfDNA in the maternal bloodstream includes cfDNA from both the mother (i.e., maternal cfDNA) and the fetus (i.e., fetal cfDNA). The fetal cfDNA originates from the placental cells undergoing apoptosis, and typically constitutes up to 25% of the total circulating cfDNA, with the balance originating from the maternal genome.

Recent technological developments have allowed for noninvasive prenatal screening of chromosomal aneuploidy in the fetus by exploiting the presence of fetal cfDNA circulating in the maternal bloodstream. Noninvasive methods relying on cfDNA sampled from the pregnant woman's blood serum are particularly advantageous over chorionic villi sampling or amniocentesis, both of which risk substantial injury and possible pregnancy loss.

Accurate determination of the fraction of fetal cfDNA taken from a maternal test sample allows for improved screening of fetal aneuploidy. The fetal fraction for male pregnancies (i.e., a male fetus) can be determined by comparing the amount of Y chromosome from the cfDNA, which can be presumed to originate from the fetus, to the amount of one or more genomic regions that are present in both maternal and fetal cfDNA. Determination of the fetal fraction for female pregnancies (i.e., a female fetus) is more complex, as both the fetus and the pregnant mother have similar sex-chromosome dosage and there are few features to distinguish between maternal and fetal DNA. Methylation differences between the fetal and maternal DNA can be used to estimate the fetal fraction of cfDNA, but such methods are often cumbersome. See, for example, Chim et al., PNAS USA, 102:14753-58 (2005). In another method, the fraction of fetal cfDNA can be determined by sequencing polymorphic loci to search for allelic differences between the maternal and fetal cfDNA. See, for example, U.S. Pat. No. 8,700,338. However, as explained in U.S. Pat. No. 8,700,338 (col. 18, lines 28-36), use of polymorphic loci to determine fetal fraction becomes unreliable when the fetal fraction drops below 3%. See also Ryan et al., Fetal Diag. & Ther., vol. 40, pp. 219-223 (Mar. 31, 2016), which describes setting a threshold for “no call” when the fetal fraction is below 2.8%.

With advancements in prenatal screening that enable detection of a greater range and complexity of genetic variation, there are increasing challenges in effectively validating clinical genomic tests and proving their analytical performances, particularly in light of the small amount of cfDNA often present in samples.

The disclosures of all publications referred to herein are each hereby incorporated herein by reference in their entireties. To the extent that any reference incorporated by references conflicts with the instant disclosure, the instant disclosure shall control.

### SUMMARY OF THE INVENTION

In one aspect, there is provided a method for evaluating and improving performance of a genetic screening test for determining a fetal chromosomal abnormality in a test chromosome in a fetus by analyzing a maternal sample of a woman carrying said fetus, wherein the maternal sample includes fetal cell-free deoxyribonucleic acid (DNA). The method may include determining a value of statistical significance for the test chromosome of each of a plurality of reference maternal samples based on measured dosages of the test chromosome in the plurality of reference maternal samples; identifying an abnormality classification for the test chromosome of each of the plurality of reference maternal samples, the abnormality classification indicating cither a positive determination or a negative determination for a fetal chromosomal abnormality in the test chromosome of a maternal sample; determining a relationship between the values of statistical significance and the abnormality classifications for the plurality of reference maternal samples; generating simulated maternal samples based on the relationship between the values of statistical significance and the abnormality classifications for the plurality of reference maternal samples; determining an abnormality classification for the test chromosome of each of the plurality of simulated maternal samples; determining a value of statistical significance predicted for the test chromosome of each of the plurality of simulated maternal samples; determining specificity values and sensitivity values for a range of abnormality classifier values for the test chromosome based on the values of statistical significance and the abnormality classifications for the plurality of reference maternal samples and the plurality of simulated maternal samples; and generating a receiver operating characteristic (ROC) curve for the genetic screening test based on the determined specificity values and sensitivity values.

In some embodiments, the abnormality classifier values for the test chromosome may represent threshold values of statistical significance above which a positive call for the fetal chromosomal abnormality in the test chromosome is indicated. In some embodiments, the method may include selecting an optimized threshold value of statistical significance for the genetic screening test based on the ROC curve by selecting a level of specificity and a level of sensitivity corresponding to a location on the ROC curve. In some embodiments, generating the ROC curve for the genetic screening test may include generating a plurality of ROC curves for the genetic screening test. In some embodiments, generating the plurality of ROC curves for the genetic screening test may include performing bootstrap resampling of the plurality of reference maternal samples and the plurality of simulated maternal samples. In some embodiments, the method may include determining average sensitivities for each specificity for the genetic screening test based on the plurality of ROC curves. In some embodiments, the method may include determining ranges of sensitivities for each specificity for the genetic screening test based on the plurality of ROC curves. In some embodiments, the method may include determining confidence intervals for sensitivities and specificities for the genetic screening test based on the plurality of ROC curves. In some embodiments, the ROC curve may represents a true-positive rate versus a false-positive rate of the genetic screening test in calling the fetal chromosomal abnormality in the test chromosome. In some embodiments, the value of statistical significance may be a Z-score, a p-value, or a probability. In some embodiments, determining the value of statistical significance for the test chromosome of each of the plurality of reference maternal samples may further include determining the value of statistical significance for the reference maternal sample based on a measured dosage, an expected dosage, and an expected variance in the number of sequencing reads per bin for the test chromosome for the reference maternal sample.

In some embodiments, the method may include determining an inferred fetal fraction of cell-free DNA in each of the plurality of reference maternal samples based on a distribution of binned reads within an interrogated region from the reference maternal sample. In some embodiments, the method may include determining hyperparameters of a prior beta distribution of unknown true fetal fractions of cell-free DNA in the plurality of reference maternal samples based on the inferred fetal fractions of cell-free DNA in the plurality of reference maternal samples. In some embodiments, the method may include generating a simulated fetal fraction for each of the simulated maternal samples based on the prior beta distribution parameterized by the determined hyperparameters. In some embodiments, the inferred fetal fraction of cell-free DNA in each of the plurality of reference maternal samples may be further determined based on whole genome sequencing reads of historical sample reads. In some embodiments, the method may include determining a relationship between the values of statistical significance and the inferred fetal fractions for the plurality of reference maternal samples. In some embodiments, the interrogated region may include at least a portion of a chromosome other than the test chromosome or the portion thereof. In some embodiments, the interrogated region may include a plurality of chromosomes.

In some embodiments, the method may include determining a sequencing read depth for each of the plurality of reference maternal samples based on a number of de-duplicated mapped reads within an interrogated region from the reference maternal sample. In some embodiments, the method may include determining a relationship between the values of statistical significance and the sequencing read depths for the plurality of reference maternal samples. In some embodiments, the method may include determining a sequencing read depth for each of the plurality of reference maternal samples based on a number of de-duplicated mapped sequencing reads within an interrogated region from the reference maternal sample. In some embodiments, the method may include determining a distribution of the values of statistical significance for the test chromosome of the plurality of reference maternal samples. In some embodiments, determining the value of statistical significance predicted for the test chromosome of each of the plurality of simulated maternal samples may include predicting the value of statistical significance for the test chromosome of each of the plurality of simulated maternal samples based on the distribution of values of statistical significance for the test chromosome of the plurality of reference maternal samples. In some embodiments, the method may include calculating an average number of sequencing reads per bin and an expected variance in the number of sequencing reads per bin for the test chromosome for each of the plurality of simulated maternal samples. In some embodiments, predicting the value of statistical significance for the test chromosome of each of the plurality of simulated maternal samples further comprises predicting the value of statistical significance for the test chromosome of each of the plurality of simulated maternal samples based on the average number of sequencing reads per bin and the variance in the number of sequencing reads per bin for the test chromosome.

In some embodiments, the method may include determining a distribution of the abnormality classifications for the plurality of reference maternal samples. In some embodiments, the distribution of the abnormality classifications for the plurality of reference maternal samples may include a multinomial distribution. In some embodiments, the multinomial distribution of the abnormality classifications for the plurality of reference maternal samples may be based on class probabilities that are obtained from a prior Dirichlet distribution. In some embodiments, determining the abnormality classification for the test chromosome of each of the plurality of simulated maternal samples may include determining the abnormality classification for the test chromosome of each of the plurality of simulated maternal samples based on the distribution of abnormality classifications for the plurality of reference maternal samples. In some embodiments, determining the relationship between the values of statistical significance and the abnormality classifications for the plurality of reference maternal samples may include determining prior distributions of a plurality of inferred latent variables related to the values of statistical significance and the abnormality classifications for the test chromosome of the plurality of reference maternal samples. In some embodiments, determining the prior distributions of the plurality of inferred latent variables related to the values of statistical significance and the abnormality classifications for the test chromosome of the plurality of reference maternal samples may include performing Markov Chain Monte Carlo sampling using sequencing reads obtained from the plurality of reference maternal samples.

In some embodiments, the method may include evaluating performance of the genetic screening test in calling a fetal chromosomal abnormality based on the ROC curve. In some embodiments, the method may include identifying a decrease in the performance of the genetic screening test in calling the fetal chromosomal abnormality in the test chromosome of the test maternal samples; and evaluating the performance of the genetic screening test based on the ROC curve. In some embodiments, the method may include identifying variance between the performance of the genetic screening test in calling the fetal chromosomal abnormality in the test chromosome of a plurality of separate sets of test maternal samples; and evaluating the performance of the genetic screening test based on the ROC curve. In some embodiments, the simulated maternal samples may represent maternal samples having a different mean sequencing depth than the plurality of reference maternal samples. In some embodiments, the method may include comparing the ROC curve for the genetic screening test to an ROC curve for another genetic screening test. In some embodiments, the dosage of the test chromosome for each of the plurality of reference maternal samples may be measured using an assay that generates a plurality of quantifiable products, wherein the number of quantifiable products in the plurality of quantifiable products indicates the measured dosage. In some embodiments, the quantifiable products may be sequencing reads. In some embodiments, the quantifiable products may be PCR products.

In some embodiments, the dosage of the test chromosome for each of the plurality of reference maternal samples may be measured by aligning sequencing reads from the test chromosome or portion thereof; binning the aligned sequencing reads in a plurality of bins; counting the number of sequencing reads in each bin; and determining an average number of reads per bin and a variation of the number of reads per bin. In some embodiments, the method may include normalizing the number of sequencing reads prior to counting the sequencing reads. In some embodiments, the fetal chromosomal abnormality may be aneuploidy. In some embodiments, the aneuploidy may be monosomy or trisomy. In some embodiments, the fetal chromosomal abnormality may be a microdeletion. In some embodiments, the test chromosome may include chromosome 13, 18, 21, X, or Y.

### BRIEF DESCRIPTION OF THE DRAWINGS

The accompanying drawings illustrate a number of example embodiments and are a part of the specification. Together with the following description, these drawings demonstrate and explain various principles of the instant disclosure.

FIG. 1 illustrates the impact of fetal fraction and assay depth (specifically sequencing read depth) on resolving a triploid test chromosome (chromosome 21 in the illustrated example) dosage and an expected test chromosome dosage (which is expected to be diploid).

FIG. 2 illustrates an exemplary workflow for the dynamic iterative depth optimization process.

FIG. 3 illustrates parameter interrelationships for an exemplary fetal fraction model.

FIG. 4 illustrates parameter interrelationships for an exemplary aneuploidy classification model for a single chromosome.

FIG. 5 illustrates parameter interrelationships for an exemplary aneuploidy classification model for multiple chromosomes.

FIG. 6A illustrates exemplary normal distributions of negative calls and positive calls made by a noninvasive prenatal screening caller.

FIG. 6B illustrates an exemplary ROC curve corresponding to the distributions of FIG. 6A.

FIG. 7 illustrates an exemplary computing system configured to perform processes described herein, including the various exemplary methods for determining a fetal chromosomal abnormality in a test chromosome or a portion thereof by analyzing a test maternal sample.

FIG. 8 illustrates a flow diagram of an exemplary method for evaluating and improving performance of a genetic screening test.

FIG. 9A illustrates exemplary ROC curves for fetal aneuploidy classification.

FIG. 9B illustrates exemplary ROC curves for fetal aneuploidy classification.

FIG. 9C illustrates exemplary ROC curves for fetal aneuploidy classification.

FIG. 10A illustrates exemplary ROC curves for fetal aneuploidy classification.

FIG. 10B illustrates exemplary ROC curves for fetal aneuploidy classification.

FIG. 10C illustrates exemplary ROC curves for fetal aneuploidy classification.

FIG. 11A illustrates a variable trajectory over the course of Markov Chain Monte Carlo (MCMC) sampling, a variable autocorrelation plot, and a histogram of trisomy positive-call frequency relative to the variable.

FIG. 11B illustrates a variable trajectory over the course of MCMC sampling, a variable autocorrelation plot, and a histogram of trisomy positive-call frequency relative to the variable.

FIG. 11C illustrates a variable trajectory over the course of MCMC sampling, a variable autocorrelation plot, and a histogram of trisomy positive-call frequency relative to the variable.

FIG. 12A illustrates estimated z-score distributions for false and true trisomy 21 calls.

FIG. 12B illustrates estimated z-score distributions for false and true trisomy 21 calls.

FIG. 13 illustrates z-score results of an exemplary new candidate caller plotted against a reference caller.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the instant disclosure covers all modifications, equivalents, and alternatives falling within the scope of the appended claims.

### DETAILED DESCRIPTION

Provided herein are methods for evaluating and improving performance of clinical genomic tests, such as prenatal screening tests used in the determination of fetal chromosomal abnormality (such as a microdeletion or chromosomal aneuploidy) in a test chromosome or a portion thereof. The methods may include analyzing a plurality of genetic screening results, such as prenatal screening results, to construct one or more receiver operating characteristic (ROC) curves. The ROC curve may be utilized to validate classifier performance of genetic screening tests. ROC curves for reference and candidate protocols may be utilized, for example, to evaluate statistically significant changes in test performance, calibrate and establish quality control thresholds for analysis, simulate hypothetical assay conditions (e.g. lower sequencing depth) to assess impact, and evaluate the variance in test performance between sequencing batches.

Noninvasive prenatal screens can be used to determine fetal aneuploidies for one or more test chromosomes using cell-free DNA from a test maternal blood sample. The results of screening can, for example, inform the patient's decision whether to pursue invasive diagnostic testing (such as amniocentesis or chronic villus sampling), which has a small (but non-zero) risk of miscarriage. Aneuploidy detection using noninvasive cfDNA analysis is linked to fetal fraction (that is, the proportion of cfDNA in the test maternal sample attributable to fetal origin). Aneuploidy can manifest in noninvasive prenatal screens that rely on a measured test chromosome dosage as a statistical increase or decrease in the count of quantifiable products (such as sequencing reads) that can be attributed to the test chromosome relative to an expected test chromosome dosage (that is, the count of quantifiable products that would be expected if the test chromosome were disomic). For samples with low fetal fraction, a large number of quantifiable products (e.g., a high read depth) are needed to achieve a statistically significant increase or decrease. Conversely, for samples with high fetal fraction, a smaller number of quantifiable products (e.g., a low read depth) can provide the statistically significant increase or decrease. The methods described herein can also be used to detect microdeletions in a fetal chromosome. Microdeletions are portions of a chromosome (often on the order of 2 million bases to about 10 million bases, but can be larger or smaller), and can cause significant deleterious effects to the fetus.

As further described herein, an initial dosage of a test chromosome or a portion thereof from a test maternal sample can be measured, and a statistical analysis (such as the determination of a value of likelihood that the test chromosome is abnormal or a value of statistical significance) can be performed. The statistical analysis can determine whether a call of normal (such as euploidy or no microdeletion) or abnormal (such as aneuploidy or the presence of a microdeletion) for the test chromosome or portion thereof can be made within the desired level of confidence. In some embodiments, if the call cannot be made within the desired level of confidence or likelihood, the chromosome dosage is re-measured using an assay that provides a higher accuracy or precision (for example, by generating a greater number of quantifiable products, such as sequencing reads). The statistical analysis can be repeated, which can reveal whether, given the subsequent statistical results, a call of normal or abnormal for the test chromosome or portion thereof can be made within the desired level of confidence.

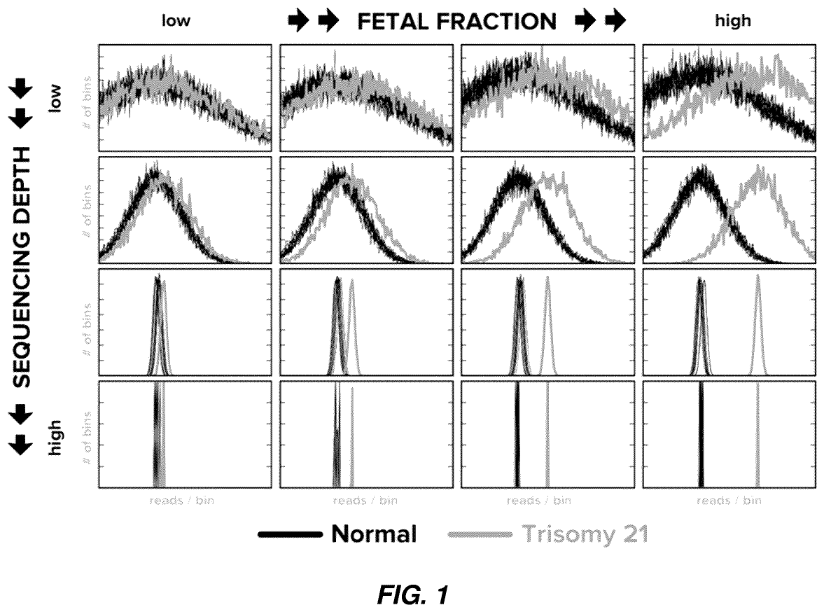

FIG. 1 illustrates the impact of fetal fraction and assay depth (specifically sequencing read depth) on resolving a triploid test chromosome (chromosome 21 in the illustrated example) dosage and an expected test chromosome dosage (which is expected to be diploid). In the example illustrated in FIG. 1, the test chromosome dosage is measured by aligning sequencing reads from the test chromosome; binning the aligned sequencing reads in a plurality of bins; counting the number of sequencing reads in each bin, including normalizing the number of sequencing reads in each bin for GC content and mappability; and determining a distribution for the number of reads per bin. The distribution for the aneuploid test chromosome and the expected distribution for the test chromosome (assuming disomy) is plotted (number of bins versus reads per bin). When the fetal fraction of cfDNA is high (right side of the figure), the sequencing depth needed to resolve the measured and expected test chromosomes is relatively low. However, when the fetal fraction of cfDNA is low (left side of figure) the sequencing depth needed to statistically distinguish the measured from the expected test chromosomes is relatively high.

Since the majority of test maternal samples will likely not require re-measurement of the test chromosome dosage, the subsequent assay may only need to be applied to a limited number of samples. By employing these methods, the cost for the noninvasive prenatal screen is more efficient (both in terms of cost and time) by minimizing the average assay depth while also yielding high sensitivity and specificity even at fetal fractions below which other noninvasive methods are able to call a normal or abnormal fetal chromosome within the desired confidence level. Because clinical guidelines recommend offering invasive diagnostic testing in the case of no-call (due to higher rates of aneuploidy in these samples), the reduced no-call rate from the methods provided herein helps reduce patient anxiety, unnecessary invasive procedures, and clinical workload burden.

Fetal fraction is influenced, in part, by the gestational age of the fetus and by the proportional size of the mother relative to the fetus. Pregnant women with a high body mass index (BMI) tend to have a lower fetal fraction at a similar gestational age. For example, women with a BMI greater than 30 are four times as likely to have a low fetal fraction of 2% to 4% (0.35 to 3.8 percentile) as women with a BMI under 30. Previous methods of noninvasive prenatal screening for aneuploidy are thus less likely to be useful for pregnant women with high BMI, or any other pregnant woman with a low fetal fraction of cfDNA. Furthermore, fetuses with chromosomal aneuploidy or certain microdeletions are more often undersized, further decreasing the fetal fraction of cfDNA. The methods described herein are more robust, and can more reliably provide screening for pregnant women with a high BMI, fetus with developmental anomalies, and at a younger gestational age. Additionally, the methods described herein may reduce the reflex rate of tested samples through improved Z-score calculations, improved background sample regressions, and utilization of clustered reference samples.

In a typical bioinformatics pipeline, such as that utilized for noninvasive prenatal screens to determine fetal aneuploidies, a variant is called positive if the confidence score (e.g., z-score, likelihood) exceeds established thresholds, which can vary depending on the type of variant, assay chemistry, and calling algorithm. Tuning this threshold and then evaluating sensitivity and specificity can be difficult (1) when reference samples with consensus genotypes are scarce and (2) when the underlying biology of the assay necessarily causes the signal of positive samples to approach the limits of detection.

To overcome this challenge, a Bayesian graphical modeling approach may be utilized. The Bayesian graphical modeling approach may be capable of deconvoluting and parameterizing the negative and positive distributions from empirical data of unlabeled samples, such as test maternal samples. One begins by postulating the interrelation between the latent distributions for variant incidence, allele fraction, and sample classification with the observed confidence score. Inference of the posterior predictive distribution—which assigns probabilistic outcome labels to the previously unlabeled data—is performed using a Markov Chain Monte Carlo (MCMC) method. We then parametrically scan a range of classification thresholds and calculate sensitivity and specificity to construct a ROC curve with confidence intervals given by bootstrap sampling.

This approach has been leveraged to evaluate major algorithmic enhancements to noninvasive prenatal screening using 34,000 random clinical samples, including >500 positives. Utilizing receiver operating characteristic (ROC) curves, it was determined that sensitivity increased significantly, with a ˜2× reduction in the false-negative rate, while maintaining 99.9% specificity. Furthermore, relationships between sequencing depth, minor allele fraction, and confidence score to decipher the limits of detection were determined. This strategy effectively determined analytical performance from a real world collection of test samples for cutting-edge genetic assays. ROC curves may thus be utilized to validate and optimize prenatal screening tests and other genetic tests to select optimal test models and to discard suboptimal test models.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

The term “average” as used herein refers to either a mean or a median, or any value used to approximate the mean or the median. An “average mean” or “average median” refers to a mean or median (or any value used to approximate the mean or the median) of the means or medians (or approximate means or medians) from a plurality of distributions. An “average variation” refers to a mean or median (or any value used to approximate the mean or the median) of variations from a plurality of distributions. An “average distribution” refers to i) an average mean or an average median, and ii) an average variation, from a plurality of distributions.

A “bin” is an arbitrary genomic region from which a quantifiable measurement can be made. When multiple bins (i.e., a plurality of bins) are subjected to common analysis, the length of each arbitrary genomic region is preferably the same and tiled across a region of interest without overlaps. Nevertheless, the bins can be of different lengths, and can be tiled across the region of interest with overlaps or gaps.

A “chromosome dosage” is a quantitated amount of a chromosome, measured directly or indirectly, or a quantitated amount of an assay product representing a chromosome. The chromosome dosage may be represented as an absolute amount or as a distribution (including a mean or median (or an approximate value representing the mean or the median) and a variation). The chromosome dosage can be an integer (such as an integer number of chromosomes or an integer number of assay products) or a fraction (such as an amount of a chromosome indirectly measured based on a quantitated amount of an assay product representing the chromosome or a normalized amount of the assay product representing the chromosome).

An “expected chromosome dosage” is a chromosome dosage that would be expected if no fetal chromosomal abnormality were present.

A “fetal chromosomal abnormality” is any chromosomal copy number variant of the fetal genome relative to the maternal genome, including a microdeletion or chromosomal aneuploidy.

An “interrogated region” is any portion of a genome, which may be contiguous or non-contiguous, and can include one or more whole chromosomes or any one or more portions of any one or more chromosomes.

A “machine-learning model” is a predictive mathematical model—which may be implemented on a computer system—that uses an observed data set of numerical or categorical data to generate a predicted outcome data set of numerical or categorical data. The model can be “trained” on a plurality of observed data sets, wherein each of the observed data sets has a known outcome data set. Once trained, the model can be applied to a novel observed data set to yield a predicted outcome data set. The term “machine learning model” includes, but is not limited to, a regression model, a linear regression model, a ridge regression model, an elastic-net model, or a random-forest model.

A “mappable” sequencing read is a sequencing read that aligns with a unique location in a genome. A sequencing read that maps to zero or two or more locations in the genome is considered not “mappable.”

A “maternal sample” refers to any sample taken from a pregnant mammal which comprises a maternal source and a fetal source of nucleic acids. The term “training maternal sample” refers to a maternal sample that is used to train a machine-learning model.

The term “maternal cell-free DNA” or “maternal cfDNA” refers to a cell-free DNA originating from a chromosome from a maternal cell that is neither placental nor fetal. The term “fetal cell-free DNA” or “fetal cfDNA” refers to a cell-free DNA originating from a chromosome from a placental cell or a fetal cell.

The term “normal” when used to characterize a putative fetal chromosomal abnormality, such as a microdeletion or aneuploidy, indicates that the putative fetal chromosomal abnormality is not present. The term “abnormal” when used to characterize a putative fetal chromosomal abnormality indicates that the putative fetal chromosomal abnormality is present.

A “variation” as used herein refers to any statistical metric that defines the width of a distribution, and can be, but is not limited to, a standard deviation, a variance, or an interquartile range.

A “value of likelihood” refers to any value achieved by directly calculating likelihood or any value that can be correlated to or otherwise indicative of likelihood. The term “value of likelihood” includes an odds ratio.

A “value of statistical significance” is any value that indicates the statistical distance of a tested event or hypothesis from a null or reference hypothesis, such as a Z-score, a p-value, or a probability.

It is understood that aspects and variations of the invention described herein include “consisting” and/or “consisting essentially of” aspects and variations.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

It is to be understood that one, some or all of the properties of the various embodiments described herein may be combined to form other embodiments of the present invention.

The section headings used herein are for organizational purposes only and are not to be construed as limiting the subject matter described.

The disclosures of all publications referred to herein are each hereby incorporated herein by reference in their entireties. To the extent that any reference incorporated by references conflicts with the instant disclosure, the instant disclosure shall control.

### Measuring Fetal Fraction

Certain regions of a genome may be over- or under- represented in the amount of fetal cell-free DNA versus maternal cell-free DNA. The amount of the over- or under-representation within these regions is proportional to the fetal fraction of cell-free DNA. Not all regions of the genome are over- or under-represented proportional to the fetal fraction of cfDNA. By binning the genome, or a portion thereof (such as an interrogated region, such as one or more chromosomes or a portion thereof), discreet portions of the genome can be isolated so that those specific regions can independently influence a machine-learning model. Measuring the amount of over- or under-representation of those regions can thus be used to indirectly measure the fetal fraction of cfDNA in a maternal sample by applying a trained machine-learning model.

In some embodiments, the fetal fraction of the cell-free DNA in a maternal sample is measured based on the over- or under-representation of fetal cell-free DNA from a plurality of bins within an interrogated region relative to maternal cell-free DNA. In some embodiments, the over- or under- representation of the fetal cell-free DNA is determined by a count of binned sequencing reads. In some embodiments, the over- or under-representation of the fetal cell-free DNA is determined by a count of binned hybridized probes.

In some embodiments, the fetal fraction of the cell-free DNA in a maternal sample is measured based on a count of binned sequencing reads from an interrogated region in the maternal sample. In some embodiments, the sequencing reads are aligned (for example, using a reference sequence), binned in a plurality of bins after being aligned, and the number of tsequencing reads in each bin are counted. In some embodiments, the counted sequencing reads are normalized, for example to account for variations in GC content or mappability of the sequencing reads. Binning of the sequencing reads isolates discrete portions of the genome so that those specific regions can independently influence the trained model.

In some embodiments, the fetal fraction of the cell-free DNA in a maternal sample is measured based on a count of binned hybridized probes from an interrogated region in the maternal sample. In some embodiments, a plurality of probes hybridize to an interrogated region, the interrogated region is binned, and the number (or density) of probes that hybridize in each bin is counted. In some embodiments, the number or density of probes is determined using a fluorescence assay. In some embodiments, the probes are bound to a microarray.

A trained machine-learning model (such as a regression model, for example a linear regression model or a ridge regression model) is used to determine the measured fetal fraction based on the number of counts (e.g., sequencing reads or hybridized probes) in each of the bins. For example, the number of counts in the bin can be used to form a bin-count vector for any given test maternal sample, which is inputted into a trained machine-learning model to determine the fetal fraction. Optionally, the trained machine-model is a ridge regression model corrected by polynomial smoothing and/or an error reduction scaling process.

The machine-learning model can be trained using a training set. The training set includes a plurality of maternal samples (i.e., training maternal samples), wherein each training maternal sample has a known fetal fraction of cell-free DNA. One or more model coefficients can be determined based on the number of counts (such as sequencing reads or hybridized probes) in each bin and the known fetal fraction for each training maternal sample in the plurality of training maternal samples. The trained model can then be applied to the test maternal sample, which can indirectly measure the fetal fraction in the test maternal sample. The known fetal fraction from the training maternal samples can be determined, for example, by relying on the proportion of Y chromosome, the methylation differential between maternal and fetal cell-free DNA, the distribution of cfDNA fragment lengths, by sequencing polymorphic loci, or by any other known method.

In some embodiments, a sequencing library from each of the training maternal samples is prepared using cell-free DNA from the pregnant woman's serum. The cell-free DNA includes both maternal cell-free DNA and fetal cell-free DNA. The sequencing library is then sequenced (for example, using massive parallel sequencing, such as on an Illumina HiSeq 2500) to generate a plurality of sequencing counts. In some embodiments, the whole genome is sequenced, and in some embodiments, a portion of the genome is sequenced. The portion of the genome can be, for example, one or more chromosomes or one or more portions of one or more chromosomes. In some embodiments, the sequencing reads are about 10 to about 1000 bases in length (such as about 10 to about 14 bases in length, about 14 to about 18 bases in length, about 18 to about 22 bases in length, about 22 to about 26 bases in length, about 26 to about 30 bases in length, about 30 to about 38 bases in length, about 38 to about 46 bases in length, about 46 to about 60 bases in length, about 60 to about 100 bases in length, about 100 to about 200 bases in length, about 200 to about 400 bases in length, about 400 to about 600 bases in length, about 600 to about 800 bases in length, or about 800 to about 1000 bases in length). In some embodiments, the sequencing reads are single-end reads and in some embodiments, the sequencing reads are paired-end reads. Sequencing paired end reads allows for the determination of the length of sequenced cell-free DNA. This information can be beneficial in training the machine-learning model, since maternal cell-free DNA is often, on average, longer than fetal cell-free DNA, and this differential can be used to determine fetal fraction. However, it has been found that training the machine-learning model using paired-end reads is not necessary, and substantial information can be gained from single-end reads alone. As single-end reads provide substantial time and cost savings, single-end reads are preferred.

The sequencing reads from an interrogated region from the training maternal samples are then aligned, for example using one or more reference sequences (such as a human reference genome). The interrogated region is those portions of the sequenced genome from the training maternal samples that are used to train the machine-learning model (e.g., the linear regression model or the ridge regression model). In some embodiments, the interrogated region is the whole genome. In some embodiments, the interrogated region excludes the X chromosome or the Y chromosome. In some embodiments, the interrogated region is one or more chromosomes, or one or more portions of one or more chromosomes. For example, the interrogated region can be a plurality of predetermined bins, which may be on the same chromosome or on different chromosomes.

The aligned sequencing reads from the interrogated region are binned in a plurality of bins. The bins are discrete regions along the genome or chromosome. Smaller bins provide higher resolution of the interrogated region. In some embodiments, the bins are about 1 base to about 1 chromosome in length, such as about 1 kilobases to about 200 kilobases in length (such as about 1 kilobases to about 5 kilobases, about 5 kilobases to about 10 kilobases, about 10 kilobases to about 20 kilobases, about 20 kilobases to about 50 kilobases, about 50 kilobases to about 100 kilobases, or about 100 kilobases to about 200 kilobases). In some embodiments, the interrogated region comprises about 100 bins to about 100,000 bins (such as between about 50 bins and about 100 bins, between about 100 bins and about 200 bins, between about 200 bins and about 500 bins, between about 500 bins and about 1000 bins, between about 1000 bins and about 2000 bins, between about 2000 bins and about 5000 bins, between about 5000 bins and about 10,000 bins, between about 10,000 bins and about 20,000 bins, between about 20,000 bins and about 40,000 bins, between about 40,000 bins and about 60,000 bins, between about 60,000 bins and about 80,000 bins, or between about 80,000 bins and about 100,000 bins). Preferably, the bins are of equal size.

The number of sequencing reads in each bin within the interrogated region for each training sample is counted. The counted sequencing reads for each bin are optionally normalized. Normalization can account for variations in GC content or mappability of the reads between the bins. For example, some bins within the interrogated region may have a higher GC content than other bins within the interrogation region. The higher GC content may increase or decrease the sequencing efficiency within that bin, inflating the relative number of sequencing reads for reasons other than fetal fraction. Methods to normalize GC content are known in the art, for example as described in Fan & Quake, PLoS ONE, vol. 5, e10439 (2010). Similarly, the certain bins within the interrogated region may be more easily mappable (or alignable to the reference interrogated region), and a number of sequencing reads may be excluded, thereby deflating the relative number of sequencing reads for reasons other than fetal fraction. Mappability at a given position in the genome can be predetermined for a given read length, k, by segmenting every position within the interrogated region into k-mers and aligning the sequences back to the interrogated region. K-mers that align to a unique position in the interrogated region are labeled “mappable,” and k-mers that no not align to a unique position in the interrogated region are labeled “not mappable.” A given bin can be normalized for mappability by scaling the number of reads in the bin by the inverse of the fraction of the mappable k-mers in the bin. For example, if 50% of k-mers within a bin are mappable, the number of observed reads from within that bin are scaled by a factor of 2. Normalization can also optionally include scaling the number of sequencing reads in each bin, for example by dividing the number of sequencing reads in each bin by the average of sequencing reads for the bins within the interrogated region.

For each training maternal sample, the numbers of sequencing reads (which may be normalized) for each bin are associated with a known fetal fraction of cell-free DNA for that training sample. The known fetal fraction may be determined using the chromosome dosage of the Y chromosome or the X chromosome (or both) of the training maternal sample. The chromosome dosage may be determined, for example, by aligning sequencing reads from the X or Y chromosome, which may be obtained simultaneously to the sequencing reads used for the interrogated regions. Because males have one Y chromosome and one X chromosome, whereas the pregnant mother has two X chromosomes and no Y chromosomes, the sequencing read density (i.e., reads per bin) of the X chromosome in male pregnancies should be (1−e/2) relative to female pregnancies, wherein e is the fetal fraction of cell-free DNA (conversely, for the Y chromosome, the sequencing read density is (1+e/2)). The fetal dosage may be determined, for example, using the methods described in Fan & Quake, PLoS ONE, vol. 5, e10439 (2010) or U.S. Patent App. No. US 2010/0112575. In some embodiments, the sequencing reads for the X chromosome or the Y chromosome are aligned (for example, using a reference X chromosome or reference Y chromosome), the aligned sequencing reads are binned, and the number of sequencing reads in each bin are counted. In some embodiments, the numbers of sequencing reads are normalized, for example to account for variations in GC content or mappability. In some embodiments, the numbers of sequencing reads are scaled, for example by dividing by the average or median number of sequencing reads. In some embodiments, the fetal fraction is determined on the basis of the Y chromosome and the X chromosome separately. In some embodiments, to account for any systematic discrepancies between the calculation of fetal fraction from the X chromosome and the Y chromosome, the general relationship between fetal fraction inferred from the Y chromosome and the fetal fraction inferred from the X chromosome is modeled using a linear fit. The slope and intercept of the linear fit is used to scale the fetal fraction inferred from the X chromosome, and the known fetal fraction is the average of the fetal fraction inferred from the Y chromosome and the scaled fetal fraction inferred from the X chromosome (it works similarly well to perform scaling on fetal fraction estimated from the Y chromosome and then average the scaled Y-chromosome fetal fraction with the X-chromosome fetal fraction). Alternative methods of determining fetal fraction for the training maternal samples include methods relying on differential methylation of the maternal and fetal cell-free DNA or polymorphic loci.

The training maternal samples are preferably derived from male pregnancies (that is, a woman pregnant with a male fetus). In some embodiments, fetal fraction determined from the Y chromosome (i.e., FF_{Y}) and fetal fraction from the X chromosome (i.e., FF_{X}) can be determined separately. Optionally, an inferred fetal fraction from the X chromosome (FF_{IX}) is determined. An inferred fetal fraction from the X chromosome is generally preferable because it can provide more accurate fetal fraction determinations. FF_{IX} can be determined by using a linear fit to model the relationship between FF_{Y} and FF_{X} for a plurality of the training maternal samples. A slope and intercept can be determined for the linear fit, and FF_{X} can be used as an independent variable to determine the dependent variable FF_{IX}. The average of FF_{Y} and FF_{IX} (or FF_{Y} and FF_{X}, if FF_{IX} is not used) can be determined, which can be used as the fetal fraction for the training maternal samples (that is, the observed fetal fraction, FF_{O}, for the training maternal samples). Although the observed fetal fraction is preferably determined using the fetal fraction determined from the X chromosome and the fetal fraction determined from the Y chromosome, in some embodiments the observed fetal fraction is determined only from the X chromosome or only from the Y chromosome.

The machine-learning model can be, for example, a regression model, such as a multivariate linear regression model or a multivariate ridge regression model. The machine-learning model can be trained to determine one or more model coefficients using the training maternal samples, each with a known fetal fraction and a vector including the sequencing read counts (which may be normalized) for the bins in the interrogation region. Exemplary linear regression models include elastic net (Enet) and reduced-rank regression with the rank estimated using the weighted rank selection criterion (WRSC), and further detailed in Kim et al., Prenatal Diagnosis, vol. 35, pp. 810-815 (2015) (including Supporting Information).

$$F {F}_{i , r e g r e s s e d} = \vec{β} * {\vec{x}}_{i} + c$$

where FF_{i,regressed} is the fetal fraction determined by the linear model, \vec{x}ᵢ is the bin-count vector for sample i, \vec{β} is a regression coefficient vector, and c is the intercept of the model. The regression coefficient and the intercept can be determined by training the machine-learning model on the training maternal samples, for example, by linear regression or ridge regression. For example, the regression coefficient and the intercept can be determined by minimizing the square error with L₂ norm regularization with magnitude α according to:

$$\vec{β} , c = arg {min}_{β , c} {\sum }_{i} {( F {F}_{i , r e g r e s s e d} - F {F}_{i ,} )}^{2} + α {‖ \vec{β} ‖}^{2}$$

In some embodiments, the process of determining the regression coefficient includes scaling the bin counts (d_{i,j}) such that the median is set to 0 and the variation (e.g., the interquartile range) is set to 1 for each bin j across all training maternal samples used to train the machine-learning model (also referred to as a robust scalar transform). In some embodiments, the machine-learning model is trained using ridge regression. The ridge parameter α can be set by the user. Since the machine-learning model is underdetermined (that is, there are more bin count variables than fetal fraction outputs), the confidence in the model coefficients can be determined using a randomized k-fold validation (e.g., 10-fold validation) to iteratively determine the coefficients. For example, 90% of the training maternal samples (randomly selected) can be used for any given iteration, and the coefficients can be determined for 10 iterations with training maternal samples randomly selected for each iteration. In some embodiments, the regression model (such as a ridge regression model) is corrected by polynomial smoothing and/or an error reduction scaling process.

Polynomial smoothing of the trained machine-learning model can further improve the determined fetal fraction. Polynomial smoothing helps remove systematic bias artifacts. In some embodiments, a third-order polynomial is used to correct bias in the trained machine-learning model to arrive at a corrected fetal fraction (e.g., FF_{corrected}):

$$F {F}_{c o r r e c t : e d} = {c}_{0} + {c}_{1} F {F}_{r e g r e s s e d} + {c}_{2} F {F}_{r e g r e s s e d}^{2} + {c}_{3} F {F}_{r e}^{3}$$

In some embodiments, the fetal fraction is corrected using a scalar error reduction process (which may be employed in addition to or in place of the polynomial smoothing of the trained machine-learning model). The machine-learning model may over or under predict the regressed or corrected fetal fraction (FF_{regressed} or FF_{corrected}) of male or female pregnancies. To account for this, the regressed or corrected fetal fraction of the male or the female pregnancies can be multiplied by a scalar factor η. For example, in some embodiments, the fetal fraction for female pregnancies is under-predicted, and an inferred fetal fraction (FF_{inferred}) can be determined from the regressed or corrected fetal fraction as follows:

$$F {F}_{inferred}^{XY} = F {F}_{c o r r e c t e d}^{X Y}$$

$$F {F}_{inferred}^{XX} = η F {F}_{c o r r e c t e d}^{X X}$$

$$where : η = \frac{average ( F {F}_{c orrected}^{X Y} )}{average ( F {F}_{c o r rected}^{X X} )}$$

The average fetal fraction can be a median fetal fraction or a mean fetal fraction.

The trained machine-learning model can be used to estimate the fetal fraction of a test maternal sample. The test maternal sample may be from a woman with a male or female pregnancy. The fetal fraction of cell-free DNA in the test maternal sample is measured based on a count of binned sequencing reads from the interrogated region from the maternal sample. In some embodiments, a sequencing library is formed from the cell-free DNA from the test maternal sample. The sequencing library is then sequenced, for example using massive parallel sequencing (such as on an Illumina HiSeq 2500) to generate a plurality of sequencing counts. In some embodiments, the whole genome is sequenced, and in some embodiments, a portion of the genome is sequenced. The portion of the genome can be, for example, one or more chromosomes or one or more portions of one or more chromosomes. Preferably, the same portions of the genome of the test maternal sample are sequenced as for the training maternal samples. Further, it is preferable that the sequencing reads should be the same length as used to sequence the training maternal samples. The sequencing reads can be paired-end reads or single-end reads, although single-end reads are generally preferred for efficiency.

The sequencing reads from the interrogated region of the test maternal sample are aligned, for example using one or more reference sequences. Preferably, the same reference sequence or sequences are used to align the test maternal sample as the training maternal sample. The aligned sequencing reads from the test maternal sample are binned using the same bin characteristics (that is, number of bins, size of bins, and location of bins).

The number of sequencing reads in each bin within the interrogated region for each test maternal sample is counted. If the counted sequencing reads for each bin are normalized for the training maternal samples, then the counted sequencing reads for the test maternal samples are similarly normalized. Normalization can account for variations in GC content or mappability of the reads between the bins. Normalization can also include scaling the number of sequencing reads in each bin, for example by dividing the number of sequencing reads in each bin by the mean or median number of sequencing reads for the bins within the interrogated region.

The number of sequencing reads in each bin of the interrogated region of the test maternal sample (which may be normalized) can then be received by the trained machine-learning model (e.g., the linear regression model or the ridge regression model), which outputs the indirectly measured fetal fraction for the test maternal sample. The measured fetal fraction of the test maternal sample can be corrected using the polynomial smoothing process (e.g., the third-order polynomial determined) or the scalar error reduction using the predetermined scalar factor η. In some embodiments, the measured fetal fraction of the test sample can be the regressed fetal fraction, the corrected fetal fraction, or the inferred fetal fraction.

Accurate fetal fraction for the test maternal sample can be measured at low sequencing depth. In some embodiments, the test maternal sample is sequenced at a genome-wide sequencing depth of about 6 million sequencing reads or more (such as about 7 million sequencing reads or more, about 8 million sequencing reads or more, about 9 million sequencing reads or more, about 10 million sequencing reads or more, about 11 million sequencing reads or more, about 12 million sequencing reads or more, about 13 million sequencing reads or more, about 14 million sequencing reads or more, or about 15 million sequencing reads or more). In some embodiments, the training maternal samples are sequenced at an average genome-wide sequencing depth of about 6 million sequencing reads or more (such as about 7 million sequencing reads or more, about 8 million sequencing reads or more, about 9 million sequencing reads or more, about 10 million sequencing reads or more, about 11 million sequencing reads or more, about 12 million sequencing reads or more, about 13 million sequencing reads or more, about 14 million sequencing reads or more, or about 15 million sequencing reads or more). Genome-wide sequencing depth refers to the number of sequencing reads that are generated when the full genome is sequenced. That is, if less than the full genome is sequenced (for example, an interrogated region of only predetermined regions), then the sequencing depth can be proportionately reduced.

The machine-learning model can be trained from a database of training maternal samples. The database of training maternal samples can be static, or additional training maternal samples can be added to the database over time (for example, as further maternal samples are sequenced). The training maternal samples can also be simultaneously assayed along with the test maternal sample, for example by massive parallel sequencing of the plurality of maternal samples (including the training maternal samples and the test maternal samples). For example, a plurality of maternal samples can be sequenced in parallel. The fetal fraction of maternal samples taken from women with male pregnancies can be determined based on the dosage of the Y chromosome or X chromosome. Those maternal samples from women with male pregnancies can then be used to train a machine-learning model that is used to determine the fetal fraction of remaining maternal samples taken from women with female pregnancies. By regularly retraining the machine-learning model, the model is controlled for fluctuations in laboratory conditions.

### Measuring Chromosome Dosage

The dosage of the test chromosome or a test portion of a chromosome in the test maternal sample can be measured and compared to an expected dosage for the test chromosome (or test portion of the chromosome), where the expected dosage is the dosage if the test chromosome or portion thereof were normal (e.g., euploid or no microdeletion). Chromosome dosage can be measured, for example, using an assay that generates a plurality of quantifiable products (such as sequencing reads or PCR (such as digital PCR) products originating from the test chromosome), wherein the number of quantifiable products indicates the measured test chromosome dosage.

In some embodiments, the test chromosome or a test portion of the chromosome is selected from the maternal sample prior to generating the quantifiable products (i.e., selectively isolated from the maternal sample prior to generating the quantifiable products). Such methods for selection include, for example, selective capture (such as hybridization). In some embodiments, the quantifiable products used to measure the chromosome dosage can be selected after being generated, for example by filtering sequencing reads. In some embodiments, the quantifiable products are generated simultaneously to selecting the test chromosome or test portion of the chromosome, for example by selective PCR amplification.

The original source (i.e., fetal or maternal test chromosome) of the quantifiable products need not be distinguished, as the measured test chromosome dosage is used in conjunction with the measured fetal fraction, as explained below. Solely by way of example, if the test chromosome were chromosome 21, sequencing reads can be generated from both fetal chromosome 21 and maternal chromosome 21 in the test maternal sample. The generated sequencing reads can be treated identically and without regard to whether the origin of any particular sequencing read is fetal chromosome 21 or maternal chromosome 21.

Exemplary methods for determining chromosome dosage are described in Fan & Quake, PLoS ONE, vol. 5(5), e10439 (2010) and U.S. Pat. No. 8,008,018. Briefly, an assay can be performed to generate a plurality of quantifiable products from the test chromosome. As the fetal fraction in a maternal sample is usually relatively low, the majority of the quantifiable products that are generated will originate from the maternal cfDNA. However, a portion of the quantifiable products will originate from the fetal cfDNA. If, for example, the test chromosome from the fetal cfDNA is trisomic for the test chromosome, the number of resulting sequencing quantifiable products will be greater than would be expected if the fetal cfDNA were disomic for the test chromosome.

In some embodiments, a test portion of a chromosome is selected as a putative microdeletion. A microdeletion is a segment of chromosomal DNA missing in at least one fetal chromosome. Exemplary microdeletions include 22q11.2 deletion syndrome, 1p36 deletion syndrome, 15q11.2 deletion syndrome, 5p deletion syndrome, and 4p deletion syndrome The dosage of the portion of the chromosome with a microdeletion will be less than the expected dosage (that is, without the microdeletion). However, assuming a euploid chromosome, the remaining portions of chromosome with the putative microdeletion will have a measured dosage that is not statistically different from the expected dosage. The expected dosage can be determined, for example, from portions of the chromosome other than the putative region, or from other chromosomes or portions of other chromosomes in the genome. The microdeletion can be detected, for example, using circular binary segmentation techniques or by using a hidden Markov model search algorithm. See, for example, Zhao et al., Detection of Fetal Subchromosomal Abnormalities by Sequencing Circulating Cell-Free DNA from Maternal Plasma, Clinical Chemistry, vol. 61, pp. 608-616 (2015). For example, a sliding window along a chromosome can select a putative microdeletion and the chromosome dosage can be measured within the selected window (for example, a reads-per-bin distribution within any given window). The measured chromosome dosage of the putative microdeletion is compared to an expected dosage, and a value of likelihood of a microdeletion or a value of statistical significance can be determined, as further explained below. In some embodiments, the microdeletion is about 500,000 bases to about 15 million bases in length (for example, about 1 million to about 2 million bases in length, about 2 million to about 4 million bases in length, about 4 million to about 6 million bases in length, about 6 million to about 8 million bases in length, about 8 million to about 10 million bases in length, about 10 million to about 12 million bases in length, or about 12 million bases to about 15 million bases in length). In some embodiments, the microdeletion is more than about 15 million bases in length.

In some embodiments, the measured dosage is compared to an expected dosage (assumed normal) using statistical analysis. The statistical analysis can be used to evaluate the measured test chromosome dosage to determine a value of statistical significance (such as a Z-score, a p-value, or a probability) and/or value of likelihood that the test chromosome or portion thereof is abnormal.

In some embodiments, the dosage of the test chromosome (or portion thereof) is measured by aligning a plurality of sequencing reads from the test chromosome (or portion) in the maternal sample, binning the aligned sequencing reads in a plurality of bins, counting the number of sequencing reads in each bin, and determining a distribution for the number of reads per bin. The sequencing reads can be generated, for example, using massive parallel sequencing techniques. In some embodiments, the sequencing reads are generated using the same assay used to measure the fetal fraction of the maternal sample (that is, the sequencing reads used to measure the chromosome dosage are generated simultaneously as the sequencing reads used to measure the fetal fraction).

The sequencing reads generated from the test chromosome (or portion thereof) are aligned, for example using a reference sequence (such as a chromosome or portion from a human reference genome). The sequencing reads are then binned in a plurality of bins. In some embodiments, the bins are about 1 base to about one chromosome in length (such as about 1 kilobase to about 200 kilobases in length such as about 1 kilobases to about 5 kilobases, about 5 kilobases to about 10 kilobases, about 10 kilobases to about 20 kilobases, about 20 kilobases to about 50 kilobases, about 50 kilobases to about 100 kilobases, or about 100 kilobases to about 200 kilobases). In some embodiments, the interrogated region comprises about 1000 bins to about 100,000 bins (such as between about 1000 bins and about 2000 bins, between about 2000 bins and about 5000 bins, between about 5000 bins and about 10,000 bins, between about 10,000 bins and about 20,000 bins, between about 20,000 bins and about 40,000 bins, between about 40,000 bins and about 60,000 bins, between about 60,000 bins and about 80,000 bins, or between about 80,000 bins and about 100,000 bins). Preferably, the bins are of equal size.

The number of sequencing reads in each bin along the test chromosome is counted. Optionally, the counted sequencing reads for each bin are normalized, for example by accounting for variations in GC content or mappability of the reads between the bins. Normalization can also optionally include scaling the number of sequencing reads in each bin, for example by dividing the number of sequencing reads in each bin by the mean or median number of sequencing reads for the bins within the interrogated region.

A distribution of the number of reads per bin can be determined for the measured dosage. The distribution for the measured dosage can include, for example, an average (mean or median, or a value approximating a mean or a median), μₜₑₛₜ, and a variation, σₜₑₛₜ of the number of reads per bin. The variation can be, for example, a standard deviation or an interquartile range.

As chromosomal abnormality (such as aneuploidy or a microdeletion) is a relatively rare event compared to chromosomal normality (such as euploidy or no microdeletion), it can be assumed that the average dosage of each chromosome or portion thereof in a sufficiently large plurality of maternal samples reflects the expected dosage (i.e., normal for each chromosome or portion thereof). In some embodiments, the plurality of maternal samples comprises a plurality of external maternal samples. In some embodiments, the plurality of maternal samples comprises a plurality of external maternal samples and the test maternal sample.

The expected dosage (that is, assuming the test chromosome is normal) for the test maternal sample can be determined based on the measured dosage of one or more external maternal samples (that is, maternal samples other than the test maternal sample) and/or the test maternal sample. For example, in some embodiments, the measured dosage of one or more chromosomes (or portions thereof) other than the test chromosome (or portion thereof) from the test maternal sample is used to determine the expected dosage of the test maternal sample (or portion thereof). In some embodiments, the measured dosage of the test chromosome (or a portion thereof) from one or more external samples is used to determine the expected dosage of the test chromosome (or portion thereof) in the test maternal sample. In some embodiments, the measured dosage of the test chromosome (or a portion thereof) from one or more external samples and the measured dosage of the test chromosome (or portion thereof) from the test maternal sample is used to determine the expected dosage of the test chromosome (or portion thereof) in the test maternal sample. In some embodiments, the measured chromosome dosage of one or more chromosomes or portion thereof (which may or may not comprise the test chromosome or portion thereof) from one or more external maternal samples is used to determine the expected dosage of the test chromosome (or portion thereof) from the test maternal sample. In some embodiments, the measured chromosome dosage of one or more chromosomes or portion thereof (which may or may not comprise the test chromosome or portion thereof) from one or more external maternal samples and the measured chromosome dosage of one or more chromosomes or portion thereof (which may or may not comprise the test chromosome thereof) from the test maternal sample is used to determine the expected dosage of the test chromosome (or portion thereof) from the test maternal sample. In some embodiments, the one or more external maternal samples are the same as one or more of the training maternal samples used to train the machine-learning model used to determine the fetal fraction of the test maternal sample.

In some embodiments, the measured dosage includes an average number of reads per bin and a variation of the number of reads per bin. In some embodiments, the expected dosage of the test chromosome or the portion thereof is the measured dosage of the chromosome or the portion thereof other than the test chromosome or the portion thereof. Preferably, if the dosage of a portion of a chromosome is measured, the portion of the chromosome is on a different chromosome than the test chromosome portion.

In some embodiments, the expected dosage of a test chromosome or a portion thereof in a test maternal sample is determined by measuring the dosages of two or more chromosomes or portions thereof other than the test chromosome or the portion thereof in the test maternal sample. That is, the expected dosage is determined using a plurality of measured dosages (other than the test chromosome or portion thereof) internal to the test maternal sample. Each measured dosage can include an average number of reads per bin and a variation of the number of reads per bin. In some embodiments, an average distribution (or average mean or average median and an average variation) of the two or more measured dosages is determined. In some embodiments, the average distribution (or average mean or average median and average variation) is the expected dosage of the test chromosome or portion thereof. In some embodiments, the average distribution (or average mean or average median and average variation) of two or more, three or more, four or more, five or more, six or more, seven or more, eight or more, nine or more, or ten or more chromosomes or portions thereof other than the test chromosome or portion thereof is the expected chromosome dosage of the test chromosome or portion thereof. In some embodiments, the two or more chromosomes include all chromosomes other than the test chromosome or portion thereof or all autosomal chromosomes other than the test chromosome or portion thereof. In some embodiments, the test chromosome or portion thereof is further included in the average distribution to determine the expected dosage of the test chromosome or portion thereof.

In some embodiments, the expected dosage of the test chromosome or portion thereof in the test maternal sample is determined by measuring the dosage of the test chromosome or portion thereof in one or more external samples. For example, the measured dosage of the test chromosome (or portion thereof) from each of the external maternal samples can be averaged to obtain an average distribution (or average mean or average median and average variation). The average distribution determined from the measured dosages of the test chromosome from the plurality of external maternal samples can be used as the expected dosage of the test chromosome from the test maternal sample.

In some embodiments, the expected dosage of one or more chromosomes (such as a test chromosome) or a portion thereof for the test maternal sample is determined by measuring the dosage of one or more chromosomes from one or more external samples. For example, in some embodiments, the expected dosage of a test chromosome or a portion thereof for the test maternal sample is determined by training a machine-learning model using a plurality of external samples, and applying the machine-learning model to the measured dosage of one or more chromosomes or a portion thereof from the test sample. The one or more chromosomes or a portion thereof used to determine the expected dosage of the test chromosome or a portion thereof in the test sample can be all chromosomes in the genome, all autosomal chromosomes, all chromosomes in the genome excluding the test chromosome, all autosomal chromosomes excluding the test chromosome, or any portion thereof.

In some embodiments, a sequencing library from each of the training maternal samples is prepared using cell-free DNA from the pregnant woman's serum. The cell-free DNA includes both maternal cell-free DNA and fetal cell-free DNA. The sequencing library is then sequenced (for example, using massive parallel sequencing, such as on an Illumina HiSeq 2500) to generate a plurality of sequencing counts. In some embodiments, the whole genome is sequenced, and in some embodiments, a portion of the genome is sequenced. The portion of the genome can be, for example, one or more chromosomes or one or more portions of one or more chromosomes. In some embodiments, the sequencing reads are about 10 to about 1000 bases in length (such as about 10 to about 14 bases in length, about 14 to about 18 bases in length, about 18 to about 22 bases in length, about 22 to about 26 bases in length, about 26 to about 30 bases in length, about 30 to about 38 bases in length, about 38 to about 46 bases in length, about 46 to about 60 bases in length, about 60 to about 100 bases in length, about 100 to about 200 bases in length, about 200 to about 400 bases in length, about 400 to about 600 bases in length, about 600 to about 800 bases in length, or about 800 to about 1000 bases in length). In some embodiments, the sequencing reads are single-end reads and in some embodiments, the sequencing reads are paired-end reads. Sequencing paired end reads allows for the determination of the length of sequenced cell-free DNA. This information can be beneficial in training the machine-learning model, since maternal cell-free DNA is often, on average, longer than fetal cell-free DNA, and this differential can be used to determine fetal fraction. However, it has been found that training the machine-learning model using paired-end reads is not necessary, and substantial information can be gained from single-end reads alone. As single-end reads provide substantial time and cost savings, single-end reads are preferred.

The sequencing reads from an interrogated region from the training maternal samples are then aligned, for example using one or more reference sequences (such as a human reference genome). The interrogated region may include those portions of the sequenced genome from the training maternal samples that are used to train the machine-learning model. In some embodiments, the interrogated region may include the whole genome. In some embodiments, the interrogated region may exclude the X chromosome or the Y chromosome. In some embodiments, the interrogated region may be one or more chromosomes, or one or more portions of one or more chromosomes. For example, the interrogated region can be a plurality of predetermined bins, which may be on the same chromosome or on different chromosomes.

The aligned sequencing reads from the interrogated region may be binned in a plurality of bins. The bins are discrete regions along the genome or chromosome. Smaller bins provide higher resolution of the interrogated region. In some embodiments, the bins are about 1 base to about 1 chromosome in length (such as about 1 kilobases to about 200 kilobases in length (such as about 1 kilobases to about 5 kilobases, about 5 kilobases to about 10 kilobases, about 10 kilobases to about 20 kilobases, about 20 kilobases to about 50 kilobases, about 50 kilobases to about 100 kilobases, or about 100 kilobases to about 200 kilobases). In some embodiments, the interrogated region comprises about 100 bins to about 100,000 bins (such as between about 50 bins and about 100 bins, between about 100 bins and about 200 bins, between about 200 bins and about 500 bins, between about 500 bins and about 1000 bins, between about 1000 bins and about 2000 bins, between about 2000 bins and about 5000 bins, between about 5000 bins and about 10,000 bins, between about 10,000 bins and about 20,000 bins, between about 20,000 bins and about 40,000 bins, between about 40,000 bins and about 60,000 bins, between about 60,000 bins and about 80,000 bins, or between about 80,000 bins and about 100,000 bins). Preferably, the bins are of equal size.

The number of sequencing reads in each bin within the interrogated region for each training sample is counted. The counted sequencing reads for each bin are optionally normalized. Normalization can account for variations in GC content or mappability of the reads between the bins. For example, some bins within the interrogated region may have a higher GC content than other bins within the interrogation region. The higher GC content may increase or decrease the sequencing efficiency within that bin, inflating the relative number of sequencing reads for reasons other than fetal fraction. Methods to normalize GC content are known in the art, for example as described in Fan & Quake, PLoS ONE, vol. 5, e10439 (2010). Similarly, the certain bins within the interrogated region may be more easily mappable (or alignable to the reference interrogated region), and a number of sequencing reads may be excluded, thereby deflating the relative number of sequencing reads for reasons other than fetal fraction. Mappability at a given position in the genome can be predetermined for a given read length, k, by segmenting every position within the interrogated region into k-mers and aligning the sequences back to the interrogated region. K-mers that align to a unique position in the interrogated region are labeled “mappable,” and k-mers that do not align to a unique position in the interrogated region are labeled “not mappable.” A given bin can be normalized for mappability by scaling the number of reads in the bin by the inverse of the fraction of the mappable k-mers in the bin. For example, if 50% of k-mers within a bin are mappable, the number of observed reads from within that bin are scaled by a factor of 2. Normalization can also optionally include scaling the number of sequencing reads in each bin, for example by dividing the number of sequencing reads in each bin by the average of sequencing reads for the bins within the interrogated region.

### Background Regression Modelling

In some embodiments, a machine-learning model (e.g., a robust regression model) may be trained using a measured dosage of a test chromosome or portion thereof and a measured dosage of at least one chromosome or portion thereof other than the test chromosome or portion thereof in a plurality of reference maternal samples, and the machine learning model may be applied to the measured dosage of the at least one chromosome or portion thereof other than the test chromosome or portion thereof in a test maternal sample to determine the expected chromosome dosage of the test chromosome or portion thereof in the test maternal sample. For example, the machine-learning model may be trained using measured dosages of a plurality of chromosomes or all chromosomes (or portions thereof) in the plurality of reference maternal samples.

The trained model may be applied to a dosage distribution vector comprising the dosages from each of the at least one chromosome or portion thereof other than the test chromosome or portion thereof from the test maternal sample to obtain the expected dosage of the test chromosome or portion thereof. In some embodiments, the dosage distribution vector may include an average (mean or median) dosage vector and a variation dosage vector (for example, the average reads per bin can be determined independently from the variation of the number of reads per bin). In some embodiments, the plurality of maternal samples may include the test maternal sample. In some embodiments, the plurality of maternal samples may exclude the test maternal sample. In some embodiments, the at least one chromosome or portion thereof other than the test chromosome or portion thereof may include all chromosomes other than the test chromosome or portion thereof or all autosomal chromosomes other than the test chromosome or portion thereof. In some embodiments, the at least one chromosome or portion thereof other than the test chromosome may further include the test chromosome.

The machine-learning model can be trained using a training set. The training set includes a plurality of maternal samples (i.e., reference maternal samples or training maternal samples), wherein each reference maternal sample has a known dosage for a plurality of chromosomes or all chromosomes (or portions thereof) in the reference maternal sample. One or more model coefficients can be determined based on the number of counts (such as sequencing reads or hybridized probes) in each bin. The trained model can then be applied to the test maternal sample determine the dosage of the test chromosome (or portion thereof) in the test maternal sample.

The machine-learning model can be, for example, a robust regression model that is robust against outliers, such as a maximum likelihood type regression model (e.g., M-estimation or Huber regression model), a least trimmed squares regression model, a MM-estimation regression model, or any other suitable robust regression model, without limitation. The machine-learning model can be trained to determine one or more model coefficients using the reference maternal samples (i.e., training maternal sample), each with a known vector including the sequencing read counts (which may be normalized) for the bins in the interrogation region.

The machine-learning model can be trained using the bin counts (which may be normalized bin counts, or log₂ normalized bin counts) from the reference maternal samples. The machine-learning model can be, for example, based on a linear model defined by:

$${μ}_{i} = {\sum }_{j \neq i} {β}_{j} {x}_{j}$$

where μᵢ is the mean or median for the expected dosage distribution of a test chromosome of a sample i determined by the regression model, xⱼ is the bin vector for the test chromosome of sample j, and βⱼ is a regression coefficient for the test chromosome of sample j. The machine-learning model may utilize a weight function that varies based on at least one of an average number of sequencing reads per bin and a variation of the number of sequencing reads per bin for portions of the interrogated region, such as portions corresponding to particular chromosomes (or portions thereof), and the regression coefficient can be determined by minimizing the square error with L₂ norm regularization with a magnitude parameter α according to:

$$ℓ ( \vec{β} ) = α {‖ \vec{β} ‖}^{2} + {\sum }_{samples} \{ \begin{matrix}{( x - μ )}^{2} & if \frac{x - μ}{σ} < 3 \\ \vert x - μ \vert & if \frac{x - μ}{σ} \geq 3\end{matrix}$$

In some embodiments, the magnitude parameter a can be set by the user. In some embodiments, the regression model may be corrected by polynomial smoothing and/or an error reduction scaling process.

The trained machine-learning model can be used to estimate the mean or median for an expected dosage distribution of a test chromosome of a test maternal sample. The test maternal sample may be from a woman with a male or female pregnancy. In some embodiments, a sequencing library may be formed from the cell-free DNA from the test maternal sample. The sequencing library then be sequenced, for example using massive parallel sequencing (such as on an Illumina HiSeq 2500) to generate a plurality of sequencing counts. In some embodiments, the whole genome is sequenced, and in some embodiments, a portion of the genome is sequenced. The portion of the genome can be, for example, one or more chromosomes or one or more portions of one or more chromosomes. The same portions of the genome of the test maternal sample may be sequenced as for the training maternal samples. Further, the sequencing reads may be the same length as used to sequence the reference maternal samples utilized to train the machine-learning model. The sequencing reads can be paired-end reads or single-end reads, although single-end reads are generally preferred for efficiency.

The sequencing reads from the interrogated region of the test maternal sample may be aligned, for example using one or more reference sequences. The same reference sequence or sequences may be used to align the test maternal sample as the training maternal sample. The aligned sequencing reads from the test maternal sample may be binned using the same bin characteristics (that is, number of bins, size of bins, and location of bins).

The number of sequencing reads in each bin within the interrogated region for each test maternal sample may be counted. If the counted sequencing reads for each bin are normalized for the reference maternal samples, then the counted sequencing reads for the test maternal samples may be similarly normalized. Normalization can account for variations in GC content or mappability of the reads between the bins. Normalization can also include scaling the number of sequencing reads in each bin, for example by dividing the number of sequencing reads in each bin by the mean or median number of sequencing reads for the bins within the interrogated region. The number of sequencing reads in each bin of the interrogated region of the test maternal sample (which may be normalized) can then be received by the trained machine-learning model (e.g., the linear regression model or the ridge regression model), which outputs an expected dosage for the test chromosome (or portion thereof) for the test maternal sample.

### Depth-Scaled Statistical Analysis

A statistical test (such as a Z-test) can be used to determine whether the measured dosage is statistically different from the expected dosage (i.e., the normal chromosome null hypothesis). To conduct the statistical test, a value of statistical significance is determined and compared to a predetermined threshold. If the value of statistical significance is above the predetermined threshold, the null hypothesis (that is, that the test chromosome is normal) can be rejected.

In some embodiments, the value of statistical significance is a Z-score. In some embodiments, the Z-score is determined using the following formula:

$$Z = \frac{{x}_{test} - {μ}_{e x p}}{{σ}_{e x p}}$$

where xₜₑₛₜ is the mean or median for the measured dosage distribution of the test chromosome (or portion thereof), μₑₓₚ is the mean or median for the expected dosage distribution, and σₑₓₚ is the variation (such as standard deviation or interquartile range) for the expected dosage distribution.

The value of statistical significance is highly correlated with fetal fraction for an aneuploid test chromosome in the test maternal sample. That is, among maternal samples that are abnormal for the test chromosome (or portion thereof), those maternal samples with a higher fetal fraction of cfDNA will have a higher absolute value of statistical significance. However, for those maternal samples with normal test chromosome, the value of statistical significance does not substantially change for differences in fetal fraction. Thus, maternal samples having low fetal fraction and abnormal test chromosome (or portion thereof) may have a value of statistical significance near those maternal samples having a normal test chromosome (or portion thereof), particularly when the sequencing depth is low. Thus, a value of likelihood that the fetal cell-free DNA is abnormal for the test chromosome can be determined based on the measured test chromosome dosage and the expected test chromosome dosage (for example, by using the Z-score), as well as the fetal fraction. This value of likelihood can be expressed as, for example an odds ratio that the test chromosome (or portion thereof) is abnormal versus normal. See, for example, U.S. Pat. No. 8,700,338.

The Z-score for a sample may vary with assay sampling depth. For example, a variation used to calculate the Z-score may decrease with increasing sampling depth, resulting in lower Z-scores for test maternal samples assayed at a lower sampling depth. The background used for determining the variation σₑₓₚ for the expected dosage distribution may also affect the Z-score. In some embodiments, the Z-score may be determined by calculating a depth-scaled variation σ_{d} that is correlated to the sampling depth of the test maternal sample. Additionally or alternatively, the Z-score may be determined by calculating a cohort-based variation σ_{c} using a plurality of cohort reference maternal samples that are selected based on one or more similarities between the cohort reference maternal samples and the test maternal sample.

In some embodiments, the Z-score may be determined for a test chromosome of a test maternal sample using a model that takes into account both depth-scaled variation σ_{d} and cohort-based variation σ_{c}. For example, the Z-score may be determined according to the following formula:

$${σ}_{e x p}^{2} = {σ}_{c}^{2} + \frac{{σ}_{d}^{2}}{d}$$

where σ_{c} is the cohort-based variation (such as standard deviation or interquartile range) for the plurality of cohort reference maternal samples, σ_{d} is the depth-scaled variation (such as standard deviation or interquartile range) for a population of reference maternal samples assayed at a particular sequencing depth or range of sequencing depths, and d is the sequencing depth of the test maternal sample.

In some embodiments, σₑₓₚ may be determined for the test chromosome (or portion thereof) based on a maximum likelihood parameter estimation model. For example, σ_{c} may be determined from the measurements of the cohort reference maternal samples according to:

$${σ}_{d}^{2} = \frac{1}{N} {\sum }_{i ∈ c o h o r t} {( {x}_{i} - {μ}_{i} )}^{2} \frac{1 - {d}_{i} 〈 {d}_{i}^{- 1} 〉}{1 - 〈 {d}_{i} 〉 〈 {d}_{i}^{- 1} 〉}$$

where xᵢ is the mean or median for the measured dosage distribution of reference maternal sample i of the plurality of cohort reference maternal samples, μᵢ is the mean or median for the expected dosage distribution of sample i, and dᵢ is the measured sequencing depth of sample i. Because the plurality of cohort reference maternal samples is selected based on similarities in characteristics of the test maternal sample and reference maternal samples of the plurality of cohort reference maternal samples, σ_{c} may be more closely correlated to each individual test maternal sample and may vary from test maternal sample to test maternal sample based on the corresponding plurality of cohort reference maternal samples selected. In at least one embodiment, σ_{c} may be determined based on variations calculated for the plurality of cohort reference maternal samples, regardless of the sequencing depths to which the cohort reference maternal samples were assayed.

In some embodiments, σ_{d} may be determined for a particular sequencing depth from a population of reference maternal samples according:

$${σ}_{d}^{2} = \frac{1}{N} {\sum }_{i} {( {x}_{i} - {μ}_{i} )}^{2} \frac{{d}_{i} - 〈 {d}_{i} 〉}{1 - 〈 {d}_{i} 〉 ( {d}_{i}^{- 1} 〉}$$

where xᵢ is the mean or median for the measured dosage distribution of sample i, μᵢ is the mean or median for the expected dosage distribution of sample i, and dᵢ is the measured sequencing depth of sample i. The population of reference maternal samples used to calculate σ_{d} may be larger than the plurality of cohort reference maternal samples used to calculate σ_{c} and may include some or all of the reference maternal samples included in the plurality of cohort reference maternal samples.

In some embodiments, σ_{d} may be calculated in advance for various chromosomes at each of a plurality of depths. For example, a large population of reference maternal samples (e.g., thousands of references samples, tens of thousands of reference maternal samples, or more) measured at various depths may be used to calculate σ_{d} for one or more test chromosomes (or portions thereof) at a plurality of leaching depths. As an example, Table 1 summarizes a plurality of exemplary predetermined σ_{d} values corresponding to various chromosomes and sequencing depths.

TABLE 1
Exemplary σ_{d} values determined for chromosomes 13-22 at various
sequencing depths.

| | Sequencing Depth | | |
|---|---|---|---|
| Chromosome | 15 mil | 30 mil | 45 mil |
| chr13 | 0.003766 | 0.003251 | 0.003061 |
| chr14 | 0.003405 | 0.002996 | 0.002846 |
| chr15 | 0.004173 | 0.003922 | 0.003835 |
| chr16 | 0.004274 | 0.003637 | 0.003397 |
| chr17 | 0.006049 | 0.005819 | 0.00574 |
| chr18 | 0.00412 | 0.003533 | 0.003314 |
| chr20 | 0.004252 | 0.003781 | 0.00361 |
| chr21 | 0.005442 | 0.004444 | 0.004057 |
| chr22 | 0.006158 | 0.005603 | 0.005405 |

In some embodiments, an expected variation determined based on σ_{c} and σ_{d} may be utilized to determine a Z-score for a test chromosome of a test maternal sample assayed at a particular sequencing depth or range of sequencing depths with greater accuracy. For example, the Z-score for the test chromosome may be determined based on an expected variation value that is calculated using a σ_{d} a that is correlated to a number of sequencing reads obtained from an assay of the test maternal sample and a σ_{c} that is calculated based on a plurality of cohort reference maternal samples having one or more similarities to the test maternal sample.

In at least one embodiment, the expected variation determined based on σ_{c} and σ_{d} may be utilized to determine the effect on the Z-score of increasing the assay depth for a test maternal sample. For example, correlations between expected variations at different sequencing depths for a test chromosome may be used to predict a resulting Z-score for a test maternal sample if the test maternal sample were sequenced using a higher depth assay. In some examples, a predicted Z-score for the test chromosome or the portion thereof of the test maternal sample may be determined based on an additional depth-scaled variation value σ_{d} that is correlated to a higher number of sequencing reads that is higher than a number of sequencing reads obtained from the test maternal sample.

### Reference Sample Clustering

In some embodiments, reference maternal samples utilized in chromosomal abnormality determinations, as described herein, may be selected based on similarities between the reference maternal samples and the test maternal samples. For example, out of a broader population of reference maternal samples, a group of reference maternal samples that are most like the test maternal sample may be utilized in analysis of the test maternal sample. Similarities in one or more characteristics of the test maternal sample and the reference maternal samples may be used to identify a suitable sub-set of reference maternal samples (i.e., cohort reference maternal samples). Reference maternal samples selected in this manner may be used for determining any suitable manner for chromosomal abnormality determinations according to any of the methods described herein. In some embodiments, values of statistical significance (such as Z-scores) may be determined, at least in part, for a test maternal sample using reference maternal samples selected based on similarities to the test maternal sample. In at least one embodiment, cohort reference maternal samples for a test maternal sample may be identified based on similarities to the test maternal sample and may be utilized to calculate a cohort-based variation value, σ_{c}, used in the calculation of expected variation value, σₑₓₚ used in the calculation of Z-scores. In some embodiments, cohort reference samples may be utilized in robust regression modeling to determine a mean or median, μₑₓₚ, for the expected dosage distribution.

In some embodiments, a broad population of reference maternal samples may be analyzed and clustered (e.g., k-means clustering or hierarchical clustering) to generate a number of clusters that each include a plurality of reference maternal samples. In some embodiments, reference maternal samples may be clustered through k-means clustering based on identified characteristics of reference maternal samples. In some embodiments, a predetermined number of clusters may be generated from a population of reference maternal samples and the clusters may each include a specified number of reference maternal samples. In some embodiments, centroids of the clusters may be identified. For each of the clusters, a specified number of reference maternal samples closest to a centroid of the cluster may be determined to be in the cluster. In some embodiments, at least some reference maternal samples may be located within more than one cluster. In some embodiments, clusters utilized in analysis of a test maternal sample may be selected based on the proximity of the centroids for the clusters to the test maternal sample. For example, clusters may be selected based on proximities of the clusters to the test maternal sample and/or based on one or more clusters being located closest to the test maternal sample.

Any suitable characteristics of reference maternal samples may be utilized in clustering the reference maternal samples. For example, characteristics of sequencing reads obtained from each of the reference maternal samples may be used to generate clusters. In some embodiments, k-means clusters may be generated based on Z-scores of various measurable metrics for the reference maternal samples. In at least one embodiment, clusters may be generated based on Z-scores of values for the reference maternal samples related to GC bias, binned sequencing depth, normalized chromosomal median, and/or any other suitable metrics. Reference maternal samples may be clustered once or periodically. In some embodiments, reference maternal samples may be clustered dynamically during testing of test maternal samples.

### Likelihood Determinations

A value of likelihood that the fetal cell-free DNA in the test maternal sample is abnormal (for example, aneuploid or has a microdeletion) for the test chromosome or test portion thereof can be determined based on the measured dosage of the test chromosome or portion thereof, the expected dosage of the test chromosome, and the measured fetal fraction. In some embodiments, the value of likelihood is determined by determining a value of statistical significance (such as a Z-score) for the test chromosome (or portion thereof) based on the measured dosage and the expected dosage; and then determining the value of likelihood of abnormality based on the value of statistical significance and the measured fetal fraction.

The value of likelihood of an abnormal chromosome (or portion thereof) can be determined using a model assuming a normal fetal test chromosome (or portion thereof) and/or a model assuming an abnormal fetal test chromosome (or portion thereof). The models can be developed, for example, using a Monte Carlo simulation to estimate the difference between the measured test chromosome dosage and the expected chromosome dosage (which may be, for example, expressed as (μₜₑₛₜ−μₑₓₚ) or a value of statistical significance) for randomly generated maternal samples drawn from empirical samples. The empirical samples can include, for example, samples taken from verified abnormal maternal samples with known fetal fraction and samples taken from non-pregnant women (where the fetal fraction is defined as 0 and the measured test chromosome dosage equals the expected dosage). The models provide a distribution of estimated difference between the measured test chromosome dosage and the expected chromosome dosage for a specified fetal fraction.

In some embodiments, the value of likelihood for an abnormal test chromosome from the test maternal sample is expressed as an odds ratio:

$$\frac{P ( {x}_{i} \vert A )}{P ( {x}_{i} \vert E )}$$

wherein P(xᵢ|A) is the probability that the difference between the measured test chromosome or portion thereof (i) dosage (which, for example, may be expressed as (μₜₑₛₜ−μₑₓₚ) or a Z-score), xᵢ, can be attributed to aneuploidy, A, and P(xᵢ|E) is the probability that the difference between the measured test chromosome dosage (which, for example, may be expressed as (μₜₑₛₜ−μₑₓₚ) or a Z-score), xᵢ, can be attributed to euploidy, E.

In some embodiments, the value of likelihood that the fetal cell-free DNA is abnormal for the test chromosome accounts for the probability that the measured fetal proportion is reflective of a true fetal fraction. When the fetal fraction is measured using any known method or the method described herein, there is some probability that the measured fetal fraction is reflective of the true fetal fraction. The value of likelihood that the fetal test chromosome from the test maternal is abnormal can be determined using the abnormal model and/or the normal model at any given fetal fraction, but this value of likelihood can also be adjusted using a weighted average across a spectrum of possible fetal fractions, wherein the probability of aneuploidy for a given fetal fraction is weighted by the probability that the measured fetal fraction reflects the true fetal fraction. This accounting can be reflected as follows:

$$P ( {A}_{i} \vert F {F}_{m} , {x}_{i} ) = {\int }_{0}^{1} d F {F}_{t} \times P ( {A}_{i} \vert F {F}_{t} , {x}_{i} ) \times P ( F {F}_{t} \vert F {F}_{m} )$$

wherein FFₘ is the measured fetal fraction and FFₜ is the true fetal fraction. The term P(Aᵢ|FFₜ, xᵢ) represents the probability of aneuploidy relative to the summed probability of euploidy and aneuploidy. Specifically:

$$P ( {A}_{i} \vert F {F}_{t} , {x}_{i} ) = \frac{P ( {z}_{i} \vert {μ}_{i , a n e u p l oid} , {σ}_{i , a n e u p l o i d} )}{P ( {z}_{i} \vert {μ}_{i , a n e u p l o i d} , {σ}_{i , aneuploid} ) + P ( {z}_{i} \vert {μ}_{i , euploid} , {σ}_{i , euploid} )}$$

where μ_{i,euploid}=0, σ_{i,euploid}=1 (σ achieves a normalized value of 1 after dividing all un-normalized values of statistical significance (e.g., Z-scores) by the standard deviation of un-normalized statistical significance (e.g., Z-scores)), and μ_{aneuploid}, and σ_{aneuploid} are functions of fetal fraction (e.g., a linear model can be fit to a set of aneuploidy samples where both the fetal fraction and Z-score are known; thus, the mean and standard deviation of Z-scores for a particular fetal fraction can be inferred from the linear model). The probabilities themselves are calculated by noting that the values of statistical significance (e.g., Z-score) distributions are Gaussian—thus completely characterized by the mean, μ, and standard deviation, σ—and using the Gaussian probability-density function to calculate the probability of a given z-score. The probability that the measured fetal proportion is reflective of a true fetal fraction can be determined, for example, by modeling a Gaussian distribution centered on the measured fetal fraction, with the distribution determined from maternal samples with known fetal fractions. The Gaussian is fit to the distribution of observed differences between the true fetal fraction and the measured fetal fraction for a plurality of samples. The difference between the true fetal fraction and the measured fetal fraction can be measured by applying the trained machine-learning model on a set of maternal samples with known fetal fraction (such as from maternal samples with male pregnancies). The distribution of differences between the true fetal fraction and the measured fetal fraction for the set of maternal samples with male pregnancies can be fit by a Gaussian model to yield mean, μ, and standard deviation, σ_{FF}; which is then applied to the test maternal sample. Thus, to calculate P(FFₜ|FFₘ), the Gaussian probability density function can be used where the mean, μ, is set to FFₘ and the standard deviation is σ_{FF}. In some embodiments, the maternal samples used to generate the model distribution comprise the training maternal samples.

### Abnormal Chromosome Calling and Dynamic Iterative Depth Optimization

In some embodiments, the test chromosome is called as abnormal (e.g., aneuploid or microdeletion) or normal (e.g., euploid or no microdeletion) using an initially determined value of statistical significance (such as a Z-score) and/or value of likelihood of abnormality. In some embodiments, the test chromosome (or portion thereof) is not called as abnormal or normal using the initially determined value of statistical significance or value of likelihood, and the test chromosome dosage is re-measured and a subsequent value of statistical significance and/or subsequent value of likelihood is determined. The re-measured dosage of the test chromosome (or portion thereof) is re-measured using a higher accuracy assay. For example, the dosage of the test chromosome (or portion thereof) can be measured by analyzing a greater number of quantifiable products (such as sequencing reads).

In some embodiments, if the initial value of statistical significance is above a predetermined threshold, the test chromosome (or portion thereof) from the test maternal sample is called as abnormal (e.g., aneuploid or microdeletion) for the fetal cfDNA. It should be noted that when evaluating the value of statistical significance (such as a Z-score) against a predetermined threshold, the absolute value of the value is preferably considered. This is because, in some instances, the aneuploid test chromosome has only a single copy (i.e., monoploid) originating from the fetal cfDNA, whereas the test chromosome would be expected to have two copies (i.e., diploid). An example of this is Turner syndrome, wherein the fetus has monosomy X. The measured test chromosome dosage would thus be less than the expected chromosome dosage, and the Z-score could be computed as a negative value. Similarly, in the circumstance of a microdeletion, an abnormal chromosome with a microdeletion would result in a lower measured dosage than a normal chromosome without the microdeletion. Thus, it is equivalent to call the test chromosome (or portion thereof) as abnormal for the fetal cfDNA when a positive value of statistical significance (e.g., Z-score) is above a positive predetermined threshold as it is to call the test chromosome as abnormal for the fetal cfDNA when a negative value of statistical significance is below a negative predetermined threshold. However, when making a specific call of fewer copies of the test chromosome (or portion thereof) in the fetal cfDNA than the expected number of copies, such as in the case of monosomy X or a microdeletion, then the call can be made when the value of statistical significance is below a negative predetermined threshold.

When the absolute value of the statistical significance is above the predetermined threshold, the measured dosage of the test chromosome (or portion thereof) is sufficiently above (or below, the case of a negative predetermined threshold) the expected dosage that the call of abnormality (such as aneuploidy or microdeletion) can be made with the desired confidence level. The desired confidence level can be used to set the predetermined threshold. In some embodiments, the desired one-tailed confidence level (α) is about 0.05 or lower (such as about 0.025 or lower, about 0.01 or lower, about 0.005 or lower, or about 0.001 or lower). In some embodiments, the predetermined threshold for the Z-score is about 2 or higher (such as about 2.5 or higher, about 3 or higher, about 3.5 or higher, about 4 or higher, about 4.5 or higher, or about 5 or higher).

When the absolute value of the value of statistical significance is below the predetermined threshold, the measured dosage of the test chromosome or portion thereof is not sufficiently above (or below in the case of a negative predetermined threshold) the expected test chromosome dosage that the call of abnormality (e.g., aneuploidy or microdeletion) cannot be made with the desired confidence level. This might occur, for example, when the test chromosome is euploid for the fetal cfDNA, but may also occur when the test chromosome is aneuploid for the cfDNA and the accuracy or precision of the measured test chromosome dosage is not sufficient to distinguish the measured test chromosome dosage from the expected test chromosome dosage. The accuracy or precision may not be sufficient, for example, if the fetal fraction of cfDNA in the test maternal sample is low and the sequencing depth is low.

In some embodiments, a value of likelihood that the fetal cell-free DNA is abnormal (e.g., aneuploid or microdeletion) for the test chromosome (or portion thereof) is determined based on the measured dosage of the test chromosome (or portion thereof), the expected dosage, and the measured fetal fraction. The value of likelihood can be, for example, odds ratio that the test chromosome for the fetal cfDNA is abnormal versus normal. In some embodiments, if the value of likelihood that the test chromosome is abnormal is below a predetermined threshold, then the test chromosome (or portion thereof) is called as normal. If, however, the value of likelihood is above the predetermined threshold, the test chromosome (or portion thereof) is not called as normal (and may be called as abnormal if the absolute value of the value of statistical significance is above the predetermined threshold). If the test chromosome (or portion thereof) is not called as normal and is not called as abnormal (for example, if the value of statistical significance is below a predetermined threshold and the value of likelihood of abnormality is above a predetermined threshold), it is generally because the measured test chromosome dosage is not sufficiently resolved from the expected test chromosome dosage. In some embodiments, if the test chromosome is not called as abnormal or normal from the initially determined value of likelihood and/or value of statistical significance, the test chromosome dosage is re-measured by analyzing a greater number of quantifiable assay products, such as sequencing reads. In some embodiments, the predetermined threshold that that the odds ratio that the test chromosome for the fetal cfDNA is abnormal versus normal is about 0.05 or higher, about 0.1 or higher, about 0.15 or higher, about 0.20 or higher, about 0.25 or higher, or about 0.3 or higher.

As an example, the determination of a call for the test chromosome or portion thereof as normal (e.g., euploid or no microdeletion) or abnormal (e.g., aneuploid or with a microdeletion) can summarized in Table 2, wherein the arrow indicates whether the indicated value is above or below the predetermined threshold.

TABLE 2
Abnormal Test Chromosome (or Portion) Calling Logic

| Value of Statistical | Value of Likelihood | |
|---|---|---|
| Significance | of Abnormality | Call |
| ↑ | n.d. | Abnormal |
| ↓ | ↑ | No call |
| ↓ | ↓ | Normal |

“n.d.” indicates that the value of likelihood of aneuploidy need not be determined if the value of statistical significance is above the predetermined threshold.

If no call is made (for example, because the value of statistical significance is too low and the value of likelihood of an abnormality is too high), the test maternal sample can be reflexed (that is, the test chromosome is re-measured) with a greater assay depth. Optionally, if the test maternal sample is reflexed, the fetal fraction can also be re-measured with a greater assay depth. In some embodiments, the reflex equation is expressed as:

$$\begin{matrix}max \\ i ∈ ( chr 13 , chr 18 , chr 21 , chrX )\end{matrix} p ( {A}_{i} \vert F {F}_{m} , {z}_{i} ) > α$$

Evaluate the probability, p(Aᵢ|FFₘ, zᵢ), of aneuploidy of a test chromosome or portion thereof, i, for a given measured fetal fraction, FFₘ, and value of statistical significance, zᵢ, across all test chromosomes or portion thereof of interest (e.g., the set of chromosome 13, chromosome 18, chromosome 21, and chromosome X; though, this set could be expanded to include other chromosomes or portions thereof that are of interest), and take the maximum of the results. If that maximum exceeds a predetermined threshold, α, the test maternal sample should be reflexed to a higher depth of sequencing.

In some embodiments, an abnormal call or a normal call is made only if the measured fetal fraction is above a predetermined threshold. In some embodiments, the predetermined threshold is about 2% or higher (such as about 2.5% or higher, about 3% or higher, about 3.5% or higher, about 4% or higher, about 4.5% or higher, or about 5% or higher), using any of the methods to determine fetal fraction as described herein. As the measured fetal fraction can vary depending on the method used, the fetal fraction may be referenced as a percentile (for example, about 0.01% of maternal samples may have a measured fetal fraction of about 1% or less). In some embodiments, the predetermined fraction is a percentile, such as about 0.25 percentile or higher, about 0.35 percentile or higher, about 0.5 percentile or higher, about 1 percentile or higher, about 1.5 percentile or higher, about 2 percentile or higher, about 2.5 percentile or higher, about 3 percentile or higher, about 3.5 percentile or higher, about 4 percentile or higher, about 5 percentile or higher, about 6 percentile or higher, about 7 percentile or higher, or about 8 percentile or higher.

In some embodiments, the test chromosome (or portion thereof) of the fetal cfDNA is called as abnormal (e.g., aneuploid or having a microdeletion) if the value of statistical significance (e.g., Z-score) is above a predetermined threshold. In some embodiments, the test chromosome (or portion thereof) of the fetal cfDNA is called as abnormal (e.g., aneuploid or having a microdeletion) only if the value of statistical significance (e.g., Z-score) is above a predetermined threshold. In some embodiments, the test chromosome of the fetal cfDNA is called as abnormal (e.g., aneuploid or having a microdeletion) only if the fetal fraction is above a predetermined threshold.

In some embodiments, the test chromosome (or portion thereof) of the fetal cfDNA is called as normal (e.g., euploid or no microdeletion) if the value of likelihood of an abnormality is below a predetermined threshold. In some embodiments, the test chromosome (or portion thereof) of the fetal cfDNA is called as normal (e.g., euploid or no microdeletion) only if the value of likelihood of an abnormality is below a predetermined threshold. In some embodiments, the test chromosome (or portion thereof) of the fetal cfDNA is called as normal (e.g., euploid or no microdeletion) if the value of likelihood of an abnormality is below a predetermined threshold and the value of statistical significance is below a predetermined threshold. In some embodiments, the test chromosome (or portion thereof) of the fetal cfDNA is called as normal (e.g., euploid or no microdeletion) only if the value of likelihood of an abnormality is below a predetermined threshold and the value of statistical significance is below a predetermined threshold. In some embodiments, the test chromosome (or portion thereof) of the fetal cfDNA is called as normal (e.g., euploid or no microdeletion) only if the fetal fraction is above a predetermined threshold.

In some embodiments, the dosage of the test chromosome (or portion thereof) is re-measured if the value of likelihood of an abnormality is above a predetermined threshold and the value of statistical significance (such as a Z-score) is below a predetermined threshold. In some embodiments, the dosage of the test chromosome (or portion thereof) is re-measured only if the value of likelihood of an abnormality is above a predetermined threshold and the value of statistical significance (such as a Z-score) is below a predetermined threshold.

In some embodiments, the dosage of the test chromosome (or portion thereof) is re-measured using a subsequent assay that generates a subsequent plurality of quantifiable products (such as sequencing reads or PCR products) from the test chromosome. In some embodiments, the fetal fraction is also re-measured using the subsequent plurality of quantifiable products. The subsequent plurality of quantifiable products can be separately analyzed, or the quantifiable products can be analyzed in combination with the plurality of quantifiable products formed from the initial assay. The number of quantifiable products in the subsequent plurality (or the number of quantifiable products in the combination of the subsequent plurality and the initial plurality) is preferably greater than the number of quantifiable products in the initial assay. By generating a large number of quantifiable products, the accuracy and/or precision of the measured chromosome dosage can be enhanced. A subsequent value of likelihood that the fetal cell-free DNA is aneuploid for the chromosome and/or a subsequent value of statistical significance can then be determined based on the re-measured chromosome dosage.

When the dosage of the test chromosome or portion thereof is re-measured, for example by using an assay that generates a subsequent plurality of quantifiable products, wherein the number of quantifiable products used to determine the re-measured dosage is greater than the number of quantifiable products used to determine an initially measured dosage, the expected chromosome dosage is adjusted to account for the increase in the number of quantifiable products. In some embodiments, the expected chromosome dosage is re-determined using the methods described herein, but with the greater number of quantifiable products.

By way of example, the number of quantifiable products (such as sequencing reads) in the initial assay used to determine the initial test chromosome dosage (and/or fetal fraction) can be about 6 million reads or more (such as about 7 million reads or more, about 8 million reads or more, about 9 million reads or more, about 10 million reads or more, about 11 million reads or more, about 12 million reads or more, about 13 million reads or more, about 14 million reads or more, about 15 million reads or more, about 16 million reads or more, or about 17 million reads or more). The number of reads is based on genome-wide sequencing, and the number of reads can be reduced by the proportion of the genome that is actually sequenced. The number of quantifiable products used to determine the subsequent dosage of the test chromosome or portion thereof (which can be, for example, the combination of the quantifiable products from the initial assay and the subsequent assay, or from the subsequent assay alone) can be, for example, about 18 million reads or more (such as about 20 million reads or more, about 25 million reads or more, about 30 million reads or more, about 35 million reads or more, about 40 million reads or more, about 45 million reads or more, about 50 million reads or more, about 60 million reads or more, about 70 million reads or more, about 80 million reads or more, about 90 million reads or more, or about 100 million reads or more). As the cost of an assay generally increases with the number of reads, it is generally preferable to minimize the number of reads necessary in an initial or subsequent assay. By performing the initial assay for all test maternal samples and only performing the subsequent assay for those test maternal samples for which no call (either aneuploid or euploid) can be made, excess and unnecessary assays are minimized.

Calls of normal or abnormal test chromosome can be made using the subsequently determined value of statistical significance (e.g., Z-score) and/or value of likelihood of abnormality in a similar manner as for the initially determined value of statistical significance and/or value of likelihood of abnormality, except the determination is based on the re-measured dosage. Because the re-measured dosage of the test chromosome or portion thereof is determined using a larger number of quantifiable products, the accuracy of the re-measured dosage and the expected dosage is greater, and the magnitude of the expected variance is less.

In some instances, the absolute value of the subsequently determined value of statistical significance (e.g., Z-score) is below the predetermined threshold and the subsequent value of likelihood of an abnormality is above the predetermined threshold. Optionally, a no-call can be made for those samples. Alternatively, the test maternal sample can be again reflexed (that is, the dosage of the test chromosome (or a portion thereof) can be again re-measured and value of statistical significance and/or value of likelihood of an abnormality re-determined). In some embodiments, test maternal samples are reflexed one or more times, two or more times, three or more times, or four or more times.

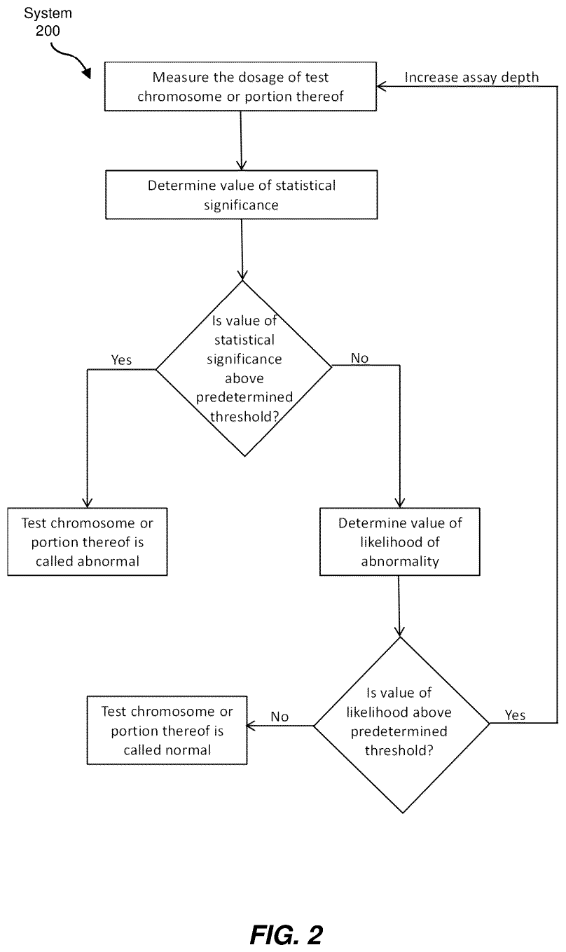

FIG. 2 illustrates one exemplary workflow for the dynamic iterative depth optimization process. An initial dosage of a test chromosome or a portion thereof is determined, for example by using an assay to generate sequencing reads, which are aligned, binned in a plurality of bins, and forming a distribution of the normalized number of reads per bin. A value of statistical significance (such as a Z-score) for the test chromosome or the portion thereof based on the measured dosage and an expected dosage. If the value of statistical significance is above a predetermined threshold, then the test chromosome or portion thereof is called as abnormal. If the value of statistical significance is below the predetermined threshold for the value of statistical significance, a value of likelihood of abnormality (such as an odds ratio) is determined. If the value of likelihood of abnormality is below a predetermined threshold for the value of likelihood, then the test chromosome or the portion thereof is called as normal. If the value of likelihood of abnormality is above the predetermined threshold for the value of likelihood, the dosage of the test chromosome or portion thereof is re-measured using an assay with increased depth (for example, using a larger number of sequencing reads). A subsequent value of statistical significance is then determined using the re-measured dosage and a re-measured expected dosage. If the value of the subsequent value of statistical significance is above the predetermined threshold for the value of statistical significance, the test chromosome or portion thereof is called as abnormal. If the value of the subsequent value of statistical significance is below the predetermined threshold for the value of statistical significance, a subsequent value of likelihood is determined. If the subsequent value of likelihood is below the predetermined threshold for the value of likelihood, the test chromosome or portion thereof is called as normal. If the subsequent value of likelihood is above the predetermined threshold for the value of likelihood, the test chromosome or portion thereof is not called or, optionally, another round of dosage measurement and statistical analysis is performed using a further increased assay depth.

In some embodiments, the call of the test chromosome (e.g., normal (such as euploid or no microdeletion), abnormal (such as aneuploid or with microdeletion), or no call) is reported (for example, to a patient, a physician, or an institution) or displayed on a monitor. In some embodiments, a value determined using any of the methods described herein (for example, a value of statistical significance (such as a Z-score), a value of likelihood (such as an odds ratio), a percent fetal fraction, or a percentile fetal fraction) is reported or displayed on a monitor.

In some embodiments, a performance summary statistic for the method (such as a sensitivity value (such as a clinical sensitivity value or an analytic sensitivity value), a specificity value (such as a clinical specificity value or an analytical specificity value), a positive predictive value, or a negative predictive value) is determined, reported (for example, to a patient, a physician, or an institution), or displayed (such as on a monitor). The performance summary statistic can be used to measure the performance of the method, which can vary based on the fetal fraction and the sequencing depth for any given test sample. For example, higher depth sequencing can result in increased sensitivity and specificity of the method. Similarly, increased fetal fraction can result in increased sensitivity and specificity of the method. In some instances (for example, when analyzing a sample with low fetal fraction), it may be preferable to report or display a call of the test chromosome (e.g., normal (such as euploid or no microdeletion, abnormal (such as aneuploid or with microdeletion)) along with one or more performance summary statistics.

In some embodiments, one or more performance summary statistics are determined based on the measured fetal fraction of cell-free DNA in the test maternal sample. For example, in some embodiments, the summary statistic is determined based on a fetal fraction range, and the measured fetal fraction is within said range. In some embodiments, the summary statistic is determined based on a specific fetal fraction consistent with the measured fetal fraction. In some embodiments, the one or more performance summary statistics (such as a clinical sensitivity value and/or clinical specificity value) determined based on the fetal fraction of the sample are determined, reported, or displayed along with the call of the test chromosome. In some embodiments, the fetal fraction is further reported or displayed along with the call and the summary statistic.

Clinical sensitivity is the fraction of condition positive samples (i.e., a population of clinical validation samples) that are identified as positive by the method when applied in clinical testing. Analytical sensitivity is the fraction of condition positive samples (i.e., a population of analytical validation samples) that are identified as positive by the method when applied to known (and validated) samples. Clinical specificity is the fraction of condition negative samples that are identified as negative by the method when applied in clinical testing. Analytical specificity is the fraction of condition negative samples that are identified as negative by the method when applied to known (and validated) samples. Clinical sensitivity and specificity are generally lower than analytical sensitivity and specificity, respectively, as the clinical statistics incorporate confounding variation in performance from both biological (e.g., confined placental mosaicism) and technical (e.g., sample preparation and handling) origins that are not represented among analytical validation samples (i.e., confounding factors). Clinical sensitivity and specificity can be determined from post-method clinical validation experiments (e.g., chorionic villi sampling or amniocentesis) of a population of clinical validation samples (for example, more than 100 samples, more than 200 samples, or more than 500 clinical validation samples).

The relationship between clinical sensitivity for the method (based on the population of clinical validation samples) can be related to the analytic sensitivity using the formula:

$$C s e n {s}_{p o p} = A s e n {s}_{p o p} - ℰ {sens}_{p o p}$$

wherein Csensₚₒₚ is the clinical sensitivity for a population of clinical validation samples, Asensₚₒₚ is the analytical sensitivity for a population of analytical validation samples, and εsensₚₒₚ is the reduction in analytical sensitivity caused by all confounding factors in the clinical validation population (such as those of biological or technical origin). Similarly, the relationship between clinical specificity for the method (based on the population of clinical validation samples) can be related to the analytic specificity using the formula:

$${Cspec}_{pop} = {Aspec}_{pop} - ε {spec}_{pop}$$

wherein Cspecₚₒₚ is the clinical specificity for a population of clinical validation samples, Aspecₚₒₚ is the analytical specificity for a population of analytical validation samples, and εspecₚₒₚ is the reduction in analytical specificity caused by all confounding factors in the clinical validation population (such as those of biological or technical origin).

Because the clinical sensitivity and clinical specificity for the method are known (or can be determined) from a clinical validation experiment, and analytical sensitivity and analytical specificity for the method are known (or can be determined) from an analytical validation experiment, the values of εsensₚₒₚ and εspecₚₒₚ can be determined. The clinical and analytical sensitivity and specificity values (that is, Csensₚₒₚ, Cspecₚₒₚ, Asensₚₒₚ, Aspecₚₒₚ) and can be determined from a population of clinical validation samples comprising a distribution of all possible fetal fractions or from a subset of fetal fractions (for example, samples with a fetal fraction of about 3% or higher, about 3.5% or higher, about 4% or higher, about 4.5% or higher, about 5% or higher, about 6% or higher, about 7% or higher or about 8% or higher), which can be used to determine εsensₚₒₚ and εspecₚₒₚ.

In some embodiments, it is assumed that the confounding factors for sensitivity and/or specificity do not vary as a function of fetal fraction. Thus, εsensₚₒₚ and εspecₚₒₚ can be considered independent of fetal fraction. Accordingly, clinical sensitivity for a subset population (for example, for samples with a specified fetal fraction or a fetal fraction within a specified fetal fraction range) can be determined according to the formula:

$${Csens}_{subset} = {Asens}_{subset} - ε {sens}_{pop}$$

wherein Csens_{subset} is the clinical sensitivity for the subset population, Asens_{subset} is the analytical sensitivity (which can be known or determined) for analytical validation samples representative of the subset population, and εsensₚₒₚ is as determined above. Similarly, clinical specificity for the subset population can be determined according to the formula:

$${Cspec}_{subset} = {Aspec}_{subset} - ε {spec}_{pop}$$

wherein Cspec_{subset} is the clinical specificity for the subset population, Aspec_{subset} is the analytical specificity (which can be known or determined) for analytical validation samples representative of the subset population, and εspecₚₒₚ is as determined above.

In some embodiments, it is not assumed that the confounding factors for sensitivity and/or specificity do not vary as a function of fetal fraction. The clinical sensitivity and clinical specificity for the subset population can then be determined by modifying the formulas above to:

$${Csens}_{subset} = {Asens}_{subset} - ( {Ksens}_{subset} \times ε {sens}_{pop} )$$

$${Cspec}_{subset} = {Aspec}_{subset} - ( {Kspec}_{subset} \times ε {spec}_{pop} )$$

wherein Ksens_{subset} and Kspec_{subset} are scaling factors to adjust the magnitude of the confounding effects on clinical sensitivity and clinical specificity, respectively, relative to the full population used to determine εsensₚₒₚ or εspecₚₒₚ as a function of the population subset (e.g., particular subset of fetal fraction or range of fetal fraction). The scaling factors Ksens_{subset} and Kspec_{subset} can be determined, for example, by in silico simulation of a large number of simulated positive or negative samples at simulated fetal fractions. The simulated samples can be called using a calling algorithm, and the frequency of the correct call is determined, yielding the analytical sensitivity and specificity for the simulated samples.

Clinical sensitivity or clinical specificity (or other summary statistic) can be determined (and reported or displayed) based on the fetal fraction of the sample. In some embodiments, the clinical sensitivity or clinical specificity (or other summary statistic) is determine for a subset population with a fetal fraction within a particular range, such as between 0% and about 7% (for example, between 0% and about 0.5%, about 0.5% and about 1%, about 1% and about 1.5%, about 1.5% and about 2%, about 2% and about 2.5%, about 3% and about 3.5%, about 3.5% and about 4%, about 4% and about 4.5%, about 4.5% and about 5%, about 5% and about 5.5%, about 5.5% and about 6%, about 6% and about 6.5%, and about 6.5% and about 7%). In some embodiments, the range of fetal fraction is within 1% or narrower (such as within 0.5% or narrower, 0.25% or narrower, or 0.1% or narrower). Solely by way of example, in some embodiments a sample with a fetal fraction of about 2.9% could be reported with a clinical sensitivity or specificity (or other summary statistic) determined for fetal fraction with a range of about 2.5% to about 3.5%, about 2.5% to about 3%, about 2.75% to about 3%, about 2.8% to about 2.9%, or about 2.9% to about 3%. In some embodiments, the clinical sensitivity or specificity (or other summary statistic) can be determine for a specific fetal fraction, for example a sample with a fetal fraction of about 2.9% could be reported or displayed with a clinical sensitivity or specificity determined for a fetal fraction of about 2.9%. For example, a distribution of clinical sensitivity or specificity (or other summary statistic) is fit to a model (such as a linear regression model) and used to determine the clinical sensitivity or specificity (or other summary statistic) for the specific fetal fraction.

The clinical sensitivity and clinical specificity (which may be determined for a particular fetal fraction or range of fetal fraction) can be used to determine other summary statistics, such as positive predictive value (PPV) or negative predictive value (NPV) of the method. By using clinical sensitivity or clinical specificity determined for fetal fraction, the positive predictive value or negative predictive value is also determined for the fetal fraction.

### Analysis Using ROC Curves

In some embodiments, classifier performance for genetic screening tests may be validated using ROC curves. In a typical bioinformatics pipeline, a variant is called positive if the confidence score (e.g., z-score, likelihood) exceeds established thresholds, which can vary depending on the type of variant, assay chemistry, and calling algorithm. Tuning this threshold and then evaluating sensitivity and specificity can be difficult (1) when reference samples with consensus genotypes are scarce and (2) when the underlying biology of the assay necessarily causes the signal of positive samples to approach the limits of detection.

An ROC curve may be determined based on few “labeled” data points. Many maternal samples may change from a positive to a negative call or from a negative to a positive call as classification thresholds are adjusted using the ROC curve. Type I error (false positive) and type II error (false negative) may be predicted. Unsupervised learning on real data (using, for example, a Gaussian mixture model) may be carried out. In some embodiments, ROC curves may be utilized as a complement to simulations.

A Bayesian graphical modeling approach capable of deconvoluting and parameterizing the negative and positive distributions from empirical data of unlabeled maternal samples may be utilized to construct ROC curves. An interrelation between latent distributions for variant incidence, allele fraction (e.g., fetal fraction), and sample classification with the observed confidence score may first be postulated. Inference of posterior predictive distributions, which assign probabilistic outcome labels to the previously unlabeled data, may be performed using Markov Chain Monte Carlo (MCMC) methodology. A range of classification thresholds may then be parametrically scanned and sensitivity and specificity may be calculated to construct an ROC curve with confidence intervals given by bootstrap sampling. These ROC curves for reference and candidate genetic screening protocols can then be used to evaluate statistically significant changes in test performance, calibrate and establish quality control thresholds for analysis, simulate hypothetical assay conditions (e.g., lower sequencing depth) to assess impact, and/or to evaluate the variance in test performance between sequencing batches.

A key variable in evaluating test performance for noninvasive prenatal screening is the ratio of signal from the fetal DNA to the noise from the maternal DNA. As discussed above, fetal fraction is the percentage of cell free DNA derived from the fetus and which may be understood and modeled from empirical data. The true fetal fraction FFₜᵣᵤₑ for a maternal sample cannot be directly observed, but there are measurements of the fetal fraction from analysis of whole-genome sequencing reads whose relationship to the true fetal fraction can be statistically modeled. For male samples, fetal fraction FF, which is the average of the fetal fraction derived from the relative decrease in chrX reads and enrichment of chrY reads, is a random variable which is statistically dependent on the FFₜᵣᵤₑ. The relationship may be given by a normal distribution with mean μ=FFₜᵣᵤₑ and a calculated standard deviation (e.g., σ=0.00317900714669, determined from variance of retesting maternal samples) according to the formula:

$$p ( FF \vert {FF}_{true} ) ∼ ( μ = {FF}_{true} , σ = 0.00317900714669 )$$

For all maternal samples the fetal fraction may be inferred from the relative distribution of reads across chromosomes other than a test chromosome of interest. This relationship may also be assumed to follow a normal distribution with mean μ=FFₜᵣᵤₑ and unknown variance σ_{inferred} according to the formula:

$$p ( {FF}_{inferred} \vert {FF}_{true} ) ∼ ( μ = {FF}_{true} , σ = {σ}_{inferred} )$$

The inverse square variance τ_{inferred}=1/σ² inferred may be drawn from a weak Gamma prior, which assumes the variance to be roughly 1.0 according to the formula:

$$p ( {τ}_{inferred} ) ∼ Gamma ( α = 100 \text{.0} , β = 0.01 )$$

The prior on the true fetal fraction itself for a sample FFₜᵣᵤₑ may be assumed to follow a beta distribution since the fetal fraction is a continuous variable bounded between 0.0 and 1.0. The two hyperparameters, α_{ff} and β_{ff}, are drawn from a uniform prior according to the formula:

$$p ( FF ) ∼ Beta ( {α}_{ff} , {β}_{ff} )$$

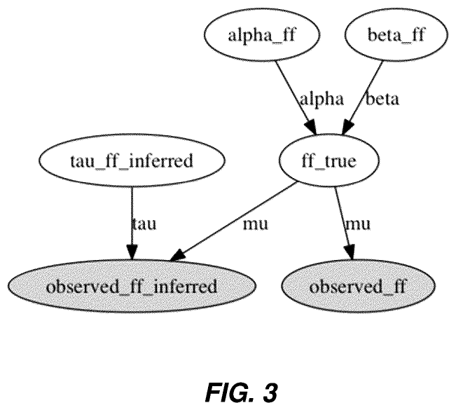

FIG. 3 shows a representation of the interrelationship between random variable FFₜᵣᵤₑ, observed values FF_{inferred} and FF (observed for male samples), and hyperparameters τ_{inferred}, α_{ff}, and β_{ff} for an exemplary fetal fraction model. The free parameters (i.e., hyperparameters) for the beta distribution may be determined by running a Maximum Posteriori (MAP) fit. The free parameters may then stored for use in analysis of noninvasive prenatal screening bioinformatics pipeline performance.

As discussed herein, noninvasive prenatal screening may be used to detect fetal aneuploidies (e.g., aneuploidies in chromosomes 13, 18, and 21 and/or monosomy in chromosome X). In some embodiments, the output of the bioinformatics pipeline may be value of statistical significance (e.g., a z-score, a p-value, or a probability statistic) for one or more test chromosomes. For example, the output of the pipeline may be a z-score statistic indicating how significantly enriched a maternal sample's normalized sequencing read depth is for a particular chromosome xᵢ when compared to the background mean μᵢ and weighted by the expected variance for such a measurement standard deviation σᵢ according to the formula:

$${z}_{i} = \frac{{x}_{i} - {μ}_{i}}{{σ}_{i}}$$

For maternal samples not having a chromosomal aneuploidy, this z-score should follow a normal distribution with mean μᵢ⁻ equal to zero and variance σᵢ. The inverse variance τ_{z} may be drawn from a weak gamma distribution prior with an expectation that the negative distribution should have unit variance by construction of the z-score in the bioinformatics pipeline algorithms according to the formula:

$$p ( {τ}_{z} ) ∼ Gamma ( α = 100 \text{.0} , β = 0.01 )$$

It is postulated that, for maternal samples having a chromosomal aneuploidy, this z-score should increase proportional to the signal to noise ratio and should thus be greater for maternal samples with greater fetal fraction and increased sequencing depth. Accordingly, the assumed functional form for the expected positive z-score may be represented by the formula:

$${μ}_{i}^{+} = {m}_{FF} {log}_{2} ( 1. + \frac{{FF}_{true}}{2.0} ) ( \frac{\sqrt{d}}{\sqrt{d} + {m}_{depth}} )$$

where m_{FF} is a random variable that captures the empirical relationship between z-score and fetal fraction. The log₂ transforms the normalization performed on the raw sequencing depth, which for a true positive, would scale proportionally with fetal fraction. Additionally, d represents a number of de-duplicated mapped reads for a maternal sample and m_{depth} is a random variable that captures the empirical relationship between z-score and depth in that, as \sqrt{d}<<m_{depth}, the signal improves linearly with the square root of sequencing depth owing to the increase in sampling of maternal sample reads, and as \sqrt{d}<<m_{depth}, the data from sequencing has been saturated and variance σᵢ contains residual systematic difference between maternal samples.

For a certain small proportion of maternal samples, the z-score may result in an anomalous no-call owing to a failure of either the sequencing assay or bioinformatics pipeline analysis. The z-score in such a case may be drawn from a uniform distribution. Consequently, the distribution for the observed z-score may be dependent on the classification of sample Xᵢ for chromosome i, which cannot be observed but must be inferred from the data, as illustrated in Table 3.

TABLE 3
Distributions and corresponding classification calls

| Result | Classification |
|---|---|
| p(zᵢ\|Xᵢ)~ [symbol] (μ = μᵢ⁺, σ = σᵢ) | Xᵢ = trisomy (positive aneuploidy call) |
| p(zᵢ\|Xᵢ)~ [symbol] (μ = 0, σ = σᵢ) | Xᵢ = wild-type (negative aneuploidy call) |
| p(zᵢ\|Xᵢ)~1.0 | Xᵢ = no-call |

This classification is itself a random variable that is drawn from a multinomial distribution with class probabilities themselves drawn from, for example, a Dirichlet distribution with weight priors alphaᵢ⁺ and alphaᵢ⁻ according to the formula:

$$p ( {X}_{i} ) ∼ Dirichlet ( {alpha}_{i}^{+} , {alpha}_{i}^{-} , 1 - {alpha}_{i}^{+} - {alpha}_{i}^{-} )$$

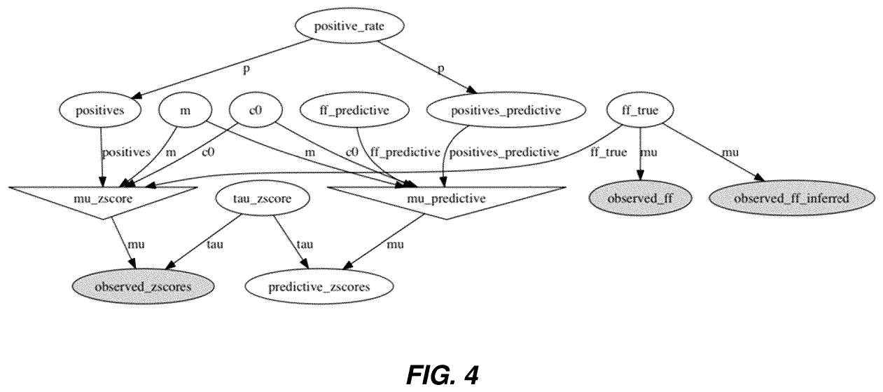

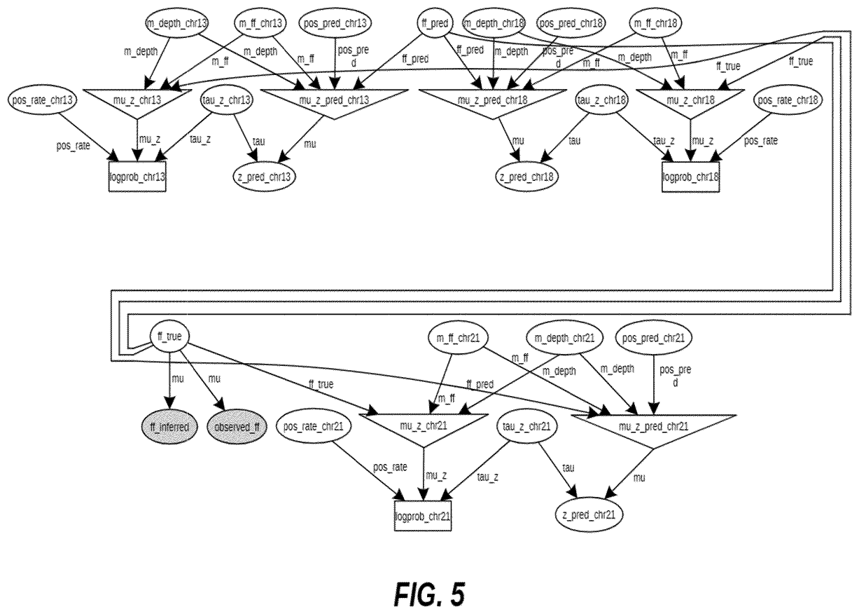

FIGS. 4 and 5 show graphical representations of the interrelationship between random variables (empty nodes), observed values (grey nodes), and hyperparameters (root nodes with no inputs) for exemplary aneuploidy classification model. FIG. 4 shows a graphical representation of interrelationships for an exemplary aneuploidy classification model for a single chromosome and FIG. 5 shows a graphical representation of interrelationships for an exemplary aneuploidy classification model for multiple chromosomes, specifically chromosomes 13, 18, and 21.

Monosomy X may be similarly modeled with a slight modification to the assumed functional form of the positive owing to the fact that a relative decrease in depth from 2 to 1 for chromosomal monosomy will have double the signal compared to a relative increase in depth from 2 to 3 for chromosomal trisomy according to the formula.

$${μ}_{i}^{+} = {m}_{FF} {log}_{2} ( 1. + \frac{{FF}_{true}}{2.0} ) ( \frac{\sqrt{d}}{\sqrt{d} + {m}_{depth}} )$$

The posterior distributions for the many latent random variables contained in the hierarchical Bayesian model may be determined through MCMC sampling with the observed empirical data using a suitable number of MCMC steps (e.g., 100,000 MCMC steps with 50,000 steps of burn-in and with data stored every 10^{th} step). The convergence of the sampling may be verified by inspecting the time correlation graphs of the random variables and confirming that all variables have been able to sample their full posterior distributions.

To this point, the interrelationship between the empirical outcomes of the bioinformatics classifier, the fetal fraction for the maternal samples, and the prevalence of positive calls for chromosomes 13, 18, 21, and X have been fully characterized. The rarity of positives in the population and the scarcity of positives in an available dataset (e.g., less than 300 positives) may present difficulty in determining false positive and false negative rates with any degree of statistical precision. At this point, additional maternal samples may be predicted or simulated by drawing from the characterized distributions themselves, which have been determined as described above. A fetal fraction for a simulated maternal sample may be obtained from the parameterized beta distribution for true fetal fraction FFₜᵣᵤₑ determined as described above. An aneuploidy classification of positive or negative for the simulated maternal sample may be obtained from the multinomial distribution for the classification of sample Xᵢ determined as described above. Resulting mean xᵢ and variance (e.g., standard deviation σᵢ) values for the z-score zᵢ for the simulated maternal sample may then be computed. A z-score zᵢ may then be predicted from the normal z-score distribution determined as described above. As a result, z-score data is paired with the aneuploidy classification for an arbitrary number of real and/or simulated maternal samples at the appropriately weighted fetal fractions and sequencing depths. This procedure can be performed on both reference and candidate datasets to create simulated z-score histograms for the positive and negative maternal samples.

Specificity may then be calculated from the predicted or simulated maternal samples by determining how many negative maternal samples will lie beyond the z-score cutoff and therefore be classified as false positives according to the following formula:

$$specificity ( {z}_{cutoff} ) = 1 - \frac{{\sum }_{j} ( {z}_{j} > {z}_{cutoff} ) \& ( {X}_{j} = negative )}{{Σ}_{j} ( {X}_{j} = negative )}$$

where Xⱼ is the true status of sample j.

Similarly, sensitivity can be calculated from those predicted or simulated maternal samples by determining how many positive maternal samples will lie below the z-score cutoff and therefore be classified as false negatives according to the following formula:

$$sensitivity ( {z}_{cutoff} ) = \frac{{\sum }_{j} ( {z}_{j} > {z}_{cutoff} ) \& ( {X}_{j} = positive )}{{Σ}_{j} ( {X}_{j} = positive )}$$

ROC curves may be constructed by parametrically scanning a range of possible z_{cutoff} values (i.e., abnormality classifier values) and then plotting the resulting sensitivity vs. (1−specificity) relationship (i.e., true-positive rate vs. false-positive rate). The statistical significance of the ROC curves can be determined by performing a bootstrap sampling of the original dataset multiple times (e.g., 10 or more times) and determining a family of ROC curves from the data. The center point, confidence intervals, and 25% and 75% percentile of sensitivity for each specificity can then be determined based on the family of ROC curves. ROC curves constructed in this manner for reference and candidate genetic screening protocols may, for example, be used to evaluate statistically significant changes in test performance, calibrate and establish quality control thresholds for analysis, simulate hypothetical assay conditions (e.g. lower sequencing depth) to assess impact, and/or to evaluate the variance in test performance between sequencing batches.

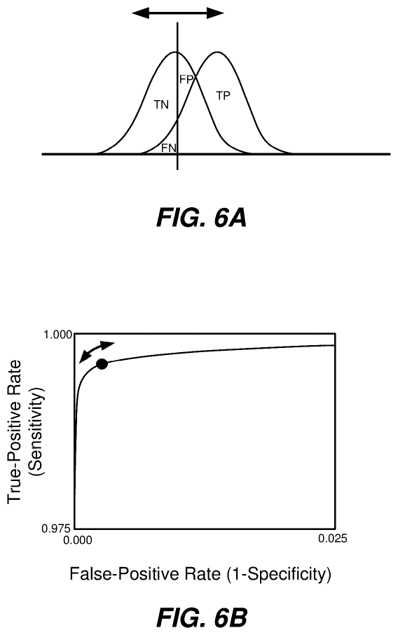

FIGS. 6A and 6B illustrate the effect of changing a classification threshold, such as a z-score threshold, using an ROC curve. FIG. 6A illustrates exemplary normal distributions of negative calls and positive calls made by a noninvasive prenatal screening caller, with true-negative (“TN”) and false-negative (“FN”) call regions shown to the left of a vertical classifier threshold line and true-positive (“TP”) and false-positive (“FP”) call regions shown to the right of the vertical classifier threshold line. FIG. 6B illustrates an exemplary ROC curve corresponding to the distributions. As the aneuploidy classifier threshold (vertical line shown in FIG. 6A) is moved, the corresponding position along the ROC curve also moves. The ROC curve may be used in this manner to determine a desired sensitivity and corresponding specificity. Maternal samples may change from a positive to a negative call or from a negative to a positive call as classification thresholds are adjusted using the ROC curve. Type I error (false positive) and type II error (false negative) may be predicted. Unsupervised learning on real data (using, for example, a Gaussian mixture model) may be carried out. In some embodiments, ROC curves may be utilized as a complement to simulations.

### Computing Systems

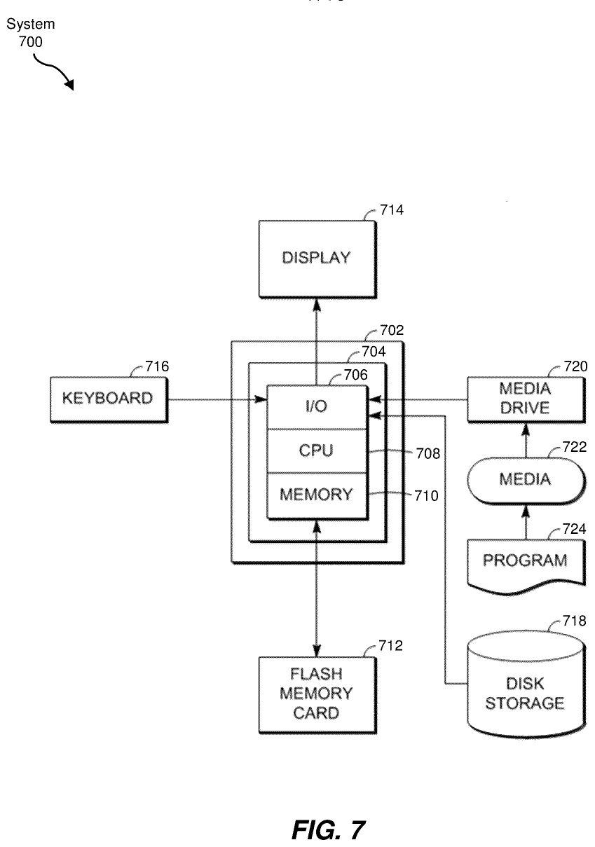

In some embodiments, the methods described herein are implemented by a program executed on a computer system. FIG. 7 depicts an exemplary computing system 700 configured to perform any one of the above-described processes, including the various exemplary methods for determining a fetal chromosomal abnormality in a test chromosome or a portion thereof by analyzing a test maternal sample. The computing system 700 may include, for example, a processor, memory, storage, and input/output devices (e.g., monitor, keyboard, disk drive, Internet connection, etc.). The computing system 700 may include circuitry or other specialized hardware for carrying out some or all aspects of the processes. For example, in some embodiments, the computing system includes a sequencer (such as a massive parallel sequencer). In some operational settings, computing system 700 may be configured as a system that includes one or more units, each of which is configured to carry out some aspects of the processes either in software, hardware, or some combination thereof.

FIG. 7 depicts computing system 700 with a number of components that may be used to perform the above-described processes. The main system 702 includes a motherboard 704 having an input/output (“I/O”) section 706, one or more central processing units (“CPU”) 708, and a memory section 710, which may have a flash memory card 712 related to it. The I/O section 706 is connected to a display 714, a keyboard 716, a disk storage unit 718, and a media drive unit 720. The media drive unit 720 can read/write a computer-readable medium 722, which can contain programs 724 and/or data.

### Exemplary Methods

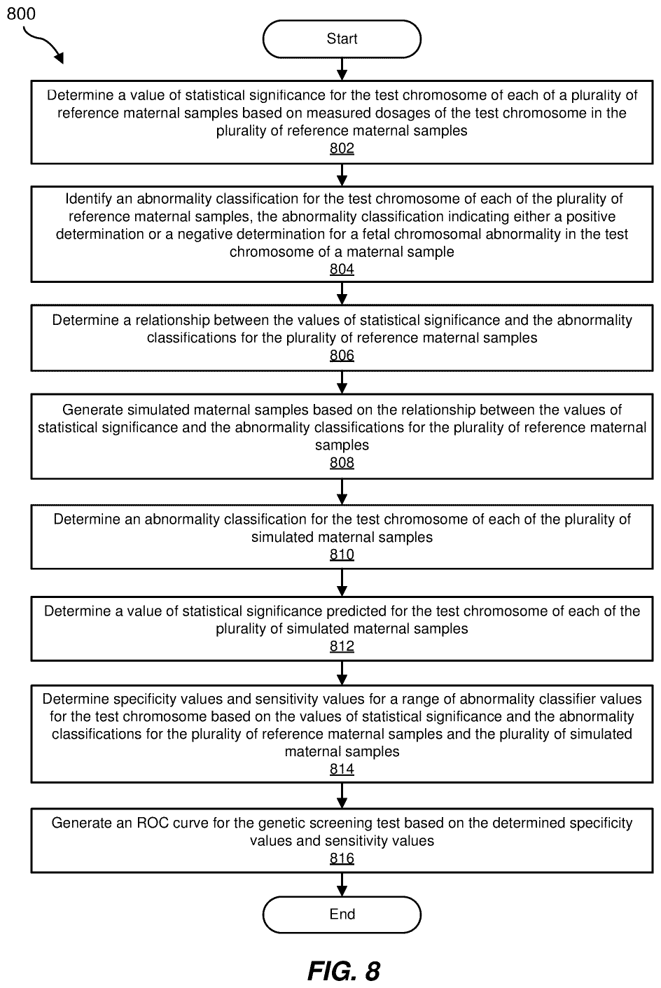

FIG. 8 is a flow diagram of an exemplary method 800 for validating genetic screening test performance in determining a chromosomal abnormality in a test chromosome. Some of the steps shown in FIG. 8 may be performed by any suitable computer-executable code and/or computing system, including system 700 in FIG. 7. In one example, some of the steps shown in FIG. 8 may represent an algorithm whose structure includes and/or is represented by multiple sub-steps, examples of which will be provided in greater detail below.

As illustrated in FIG. 8, at step 802, a value of statistical significance for the test chromosome of each of a plurality of reference maternal samples may be determined based on measured dosages of the test chromosome in the plurality of reference maternal samples. At step 804, an abnormality classification for the test chromosome of each of the plurality of reference maternal samples may be identified, the abnormality classification indicating either a positive determination or a negative determination for a fetal chromosomal abnormality in the test chromosome of a maternal sample. At step 806, a relationship between the values of statistical significance and the abnormality classifications may be determined for the plurality of reference maternal samples. At step 808, simulated maternal samples may be generated based on the relationship between the values of statistical significance and the abnormality classifications for the plurality of reference maternal samples. At step 810, an abnormality classification for the test chromosome of each of the plurality of simulated maternal samples may be determined. At step 812, a value of statistical significance predicted for the test chromosome of each of the plurality of simulated maternal samples may be determined. At step 814, specificity values and sensitivity values for a range of abnormality classifier values for the test chromosome may be determined based on the values of statistical significance and the abnormality classifications for the plurality of reference maternal samples and the plurality of simulated maternal samples. At step 816, an ROC curve for the genetic screening test may be generated based on the determined specificity values and sensitivity values.

In some embodiments, the abnormality classifier values for the test chromosome may represent threshold values of statistical significance above which a positive call for the fetal chromosomal abnormality in the test chromosome is indicated. In some embodiments, the method may include selecting an optimized threshold value of statistical significance for the genetic screening test based on the ROC curve by selecting a level of specificity and a level of sensitivity corresponding to a location on the ROC curve. In some embodiments, generating the ROC curve for the genetic screening test may include generating a plurality of ROC curves for the genetic screening test. In some embodiments, generating the plurality of ROC curves for the genetic screening test may include performing bootstrap resampling of the plurality of reference maternal samples and the plurality of simulated maternal samples. In some embodiments, the method may include determining average sensitivities for each specificity for the genetic screening test based on the plurality of ROC curves. In some embodiments, the method may include determining ranges of sensitivities for each specificity for the genetic screening test based on the plurality of ROC curves. In some embodiments, the method may include determining confidence intervals for sensitivities and specificities for the genetic screening test based on the plurality of ROC curves. In some embodiments, the ROC curve may represents a true-positive rate versus a false-positive rate of the genetic screening test in calling the fetal chromosomal abnormality in the test chromosome. In some embodiments, the value of statistical significance may be a Z-score, a p-value, or a probability. In some embodiments, determining the value of statistical significance for the test chromosome of each of the plurality of reference maternal samples may further include determining the value of statistical significance for the reference maternal sample based on a measured dosage, an expected dosage, and an expected variance in the number of sequencing reads per bin for the test chromosome for the reference maternal sample.

In some embodiments, the method may include determining an inferred fetal fraction of cell-free DNA in each of the plurality of reference maternal samples based on a distribution of binned reads within an interrogated region from the reference maternal sample. In some embodiments, the method may include determining hyperparameters of a prior beta distribution of unknown true fetal fractions of cell-free DNA in the plurality of reference maternal samples based on the inferred fetal fractions of cell-free DNA in the plurality of reference maternal samples. In some embodiments, the method may include generating a simulated fetal fraction for each of the simulated maternal samples based on the prior beta distribution parameterized by the determined hyperparameters. In some embodiments, the inferred fetal fraction of cell-free DNA in each of the plurality of reference maternal samples may be further determined based on whole genome sequencing reads of historical sample reads. In some embodiments, the method may include determining a relationship between the values of statistical significance and the inferred fetal fractions for the plurality of reference maternal samples. In some embodiments, the interrogated region may include at least a portion of a chromosome other than the test chromosome or the portion thereof. In some embodiments, the interrogated region may include a plurality of chromosomes.

In some embodiments, the method may include determining a sequencing read depth for each of the plurality of reference maternal samples based on a number of de-duplicated mapped reads within an interrogated region from the reference maternal sample. In some embodiments, the method may include determining a relationship between the values of statistical significance and the sequencing read depths for the plurality of reference maternal samples. In some embodiments, the method may include determining a sequencing read depth for each of the plurality of reference maternal samples based on a number of de-duplicated mapped sequencing reads within an interrogated region from the reference maternal sample. In some embodiments, the method may include determining a distribution of the values of statistical significance for the test chromosome of the plurality of reference maternal samples. In some embodiments, determining the value of statistical significance predicted for the test chromosome of each of the plurality of simulated maternal samples may include predicting the value of statistical significance for the test chromosome of each of the plurality of simulated maternal samples based on the distribution of values of statistical significance for the test chromosome of the plurality of reference maternal samples. In some embodiments, the method may include calculating an average number of sequencing reads per bin and an expected variance in the number of sequencing reads per bin for the test chromosome for each of the plurality of simulated maternal samples. In some embodiments, predicting the value of statistical significance for the test chromosome of each of the plurality of simulated maternal samples further comprises predicting the value of statistical significance for the test chromosome of each of the plurality of simulated maternal samples based on the average number of sequencing reads per bin and the variance in the number of sequencing reads per bin for the test chromosome.

In some embodiments, the method may include determining a distribution of the abnormality classifications for the plurality of reference maternal samples. In some embodiments, the distribution of the abnormality classifications for the plurality of reference maternal samples may include a multinomial distribution. In some embodiments, the multinomial distribution of the abnormality classifications for the plurality of reference maternal samples may be based on class probabilities that are obtained from a prior Dirichlet distribution. In some embodiments, determining the abnormality classification for the test chromosome of each of the plurality of simulated maternal samples may include determining the abnormality classification for the test chromosome of each of the plurality of simulated maternal samples based on the distribution of abnormality classifications for the plurality of reference maternal samples. In some embodiments, determining the relationship between the values of statistical significance and the abnormality classifications for the plurality of reference maternal samples may include determining prior distributions of a plurality of inferred latent variables related to the values of statistical significance and the abnormality classifications for the test chromosome of the plurality of reference maternal samples. In some embodiments, determining the prior distributions of the plurality of inferred latent variables related to the values of statistical significance and the abnormality classifications for the test chromosome of the plurality of reference maternal samples may include performing Markov Chain Monte Carlo sampling using sequencing reads obtained from the plurality of reference maternal samples.

In some embodiments, the method may include evaluating performance of the genetic screening test in calling a fetal chromosomal abnormality based on the ROC curve. In some embodiments, the method may include identifying a decrease in the performance of the genetic screening test in calling the fetal chromosomal abnormality in the test chromosome of the test maternal samples; and evaluating the performance of the genetic screening test based on the ROC curve. In some embodiments, the method may include identifying variance between the performance of the genetic screening test in calling the fetal chromosomal abnormality in the test chromosome of a plurality of separate sets of test maternal samples; and evaluating the performance of the genetic screening test based on the ROC curve. In some embodiments, the simulated maternal samples may represent maternal samples having a different mean sequencing depth than the plurality of reference maternal samples. In some embodiments, the method may include comparing the ROC curve for the genetic screening test to an ROC curve for another genetic screening test. In some embodiments, the dosage of the test chromosome for each of the plurality of reference maternal samples may be measured using an assay that generates a plurality of quantifiable products, wherein the number of quantifiable products in the plurality of quantifiable products indicates the measured dosage. In some embodiments, the quantifiable products may be sequencing reads. In some embodiments, the quantifiable products may be PCR products.

In some embodiments, the dosage of the test chromosome for each of the plurality of reference maternal samples may be measured by aligning sequencing reads from the test chromosome or portion thereof; binning the aligned sequencing reads in a plurality of bins; counting the number of sequencing reads in each bin; and determining an average number of reads per bin and a variation of the number of reads per bin. In some embodiments, the method may include normalizing the number of sequencing reads prior to counting the sequencing reads. In some embodiments, the fetal chromosomal abnormality may be aneuploidy. In some embodiments, the aneuploidy may be monosomy or trisomy. In some embodiments, the fetal chromosomal abnormality may be a microdeletion. In some embodiments, the test chromosome may include chromosome 13, 18, 21, X, or Y.

At least some values based on the results of the above-described processes can be saved for subsequent use. Additionally, a non-transitory computer-readable medium can be used to store (e.g., tangibly embody) one or more computer programs for performing any one of the above-described processes by means of a computer. The computer program may be written, for example, in a general-purpose programming language (e.g., Pascal, C, C++, Java, Python, etc.) or some specialized application-specific language.

Various exemplary embodiments are described herein. Reference is made to these examples in a non-limiting sense. They are provided to illustrate more broadly applicable aspects of the disclosed technology. Various changes may be made and equivalents may be substituted without departing from the true spirit and scope of the various embodiments. In addition, many modifications may be made to adapt a particular situation, material, composition of matter, process, process act(s) or step(s) to the objective(s), spirit or scope of the various embodiments. Further, as will be appreciated by those with skill in the art, each of the individual variations described and illustrated herein has discrete components and features that may be readily separated from or combined with the features of any of the other several embodiments without departing from the scope or spirit of the various embodiments. All such modifications are intended to be within the scope of claims associated with this disclosure.

The following non-limiting examples further illustrate the methods of the present invention. Those skilled in the art will recognize that several embodiments are possible within the scope and spirit of this invention. While illustrative of the invention, the following examples should not be construed in any way limiting its scope.

### EXAMPLES

### Example 1: ROC Curve Construction and Comparison

ROC curves were generated to evaluate and compare two different aneuploidy callers utilized in noninvasive prenatal screening for aneuploidy detection. Performance of a reference caller was compared with performance of a new candidate caller having algorithmic enhancements over the reference caller. Cell-free DNA of approximately 34,000 random clinical test maternal samples taken from pregnant women was sequenced, aligned using a reference genome, and binned in a plurality of bins, and the number of sequencing reads in each bin was counted. The test maternal samples included more than 500 positive maternal samples that were determined to include a chromosomal aneuploidy. Z-scores, fetal fractions, and resulting aneuploidy classifications (positive, negative, or no-call) for chromosomal abnormalities in chromosomes 13, 18, and 21 were respectively determined for each of the samples using the reference caller and the new caller to generate a reference caller dataset and a new caller dataset.

ROC curves for each of the reference caller dataset and the new caller dataset were respectively constructed using a hierarchical Bayesian graphical modeling approach as described herein. Simulated maternal samples were generated as described herein to determine the false-positive and false-negative aneuploidy rates with greater statistical precision. Hyperparameters for true fetal fractions FFₜᵣᵤₑ were assumed to follow a beta distribution and were determined by running a MAP fit to obtain the values shown in Table 4.

TABLE 4
MAP Fit Hyperparameter Values

| Parameter | MAP Value |
|---|---|
| τ_{ff} | 98950.2718055153 |
| τ_{inferred} | 7429.35013192 |
| α_{ff} | 4.39876319704 |
| β_{ff} | 43.2072777494 |

Classifications were drawn from a multinomial distribution with class probabilities themselves drawn from a Dirichlet distribution. The posterior distributions for latent random variables contained in the hierarchical Bayesian model were determined through MCMC sampling with the observed empirical data using 100,000 MCMC steps with 50,000 steps of burn-in and with data stored every 10^{th} step. Convergence of the sampling was verified by inspecting time correlation graphs of the random variables and confirming that all variables have been able to sample their full posterior distributions.

Additional simulated maternal samples were generated by drawing from the characterized distributions. Fetal fractions for the simulated maternal samples were obtained from the parameterized beta distribution for true fetal fraction FFₜᵣᵤₑ determined as described herein. Aneuploidy classifications of positive or negative for the simulated maternal samples was obtained from the multinomial distribution for the classification of sample Xᵢ determined. Resulting mean xᵢ and variance (e.g., standard deviation σᵢ) values for the z-score zᵢ for the simulated maternal samples was then be computed. A z-score zᵢ was then predicted from the normal z-score distribution. This procedure was performed on both the reference caller and the new caller datasets to create simulated z-score histograms for the positive and negative maternal samples.

Specificity was then calculated from the simulated maternal samples by determining how many negative maternal samples will lie beyond the z-score threshold and therefore be classified as false positives. Sensitivity was calculated from the simulated maternal samples by determining how many positive maternal samples will lie below the z-score threshold and therefore be classified as false negatives. The ROC curves for each of the reference caller and the new candidate caller were then constructed by parametrically scanning a range of possible z_{cutoff} values (i.e., abnormality classifier values) and then plotting the resulting sensitivity vs. (1−specificity) relationship (i.e., true-positive rate vs. false-positive rate). The statistical significance of the ROC curves was determined by performing a bootstrap sampling of each of the original reference caller and new caller datasets 10 times and determining a family of ROC curves from the data. The center point, confidence intervals, and 25% and 75% percentile of sensitivity for each specificity was then determined based on the family of ROC curves.

Based on the ROC curves, it was determined that sensitivity increased significantly for the new candidate caller relative to the reference caller, with approximately a two- to three-fold reduction in false-negative rate, while the specificity was maintained at or above 99%. Additionally, a relationship between sequencing depth, minor allele fraction, and confidence score was determined and utilized to determine limits of detection for the noninvasive prenatal screening. Utilizing the ROC curves effectively enabled determination of the analytical performance of the noninvasive prenatal screening based on a real-world collection of test samples.

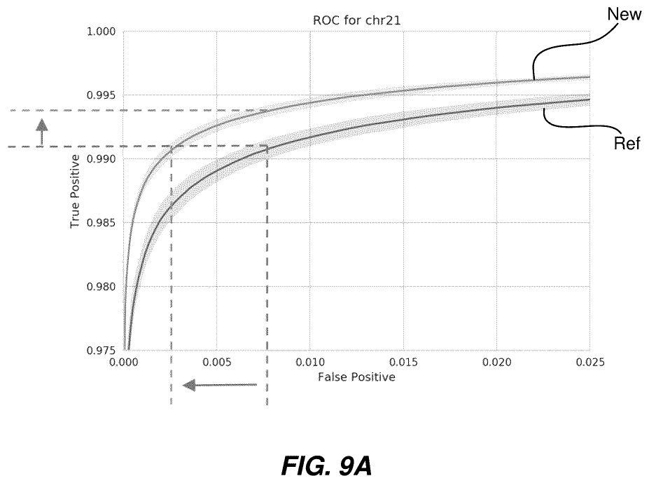

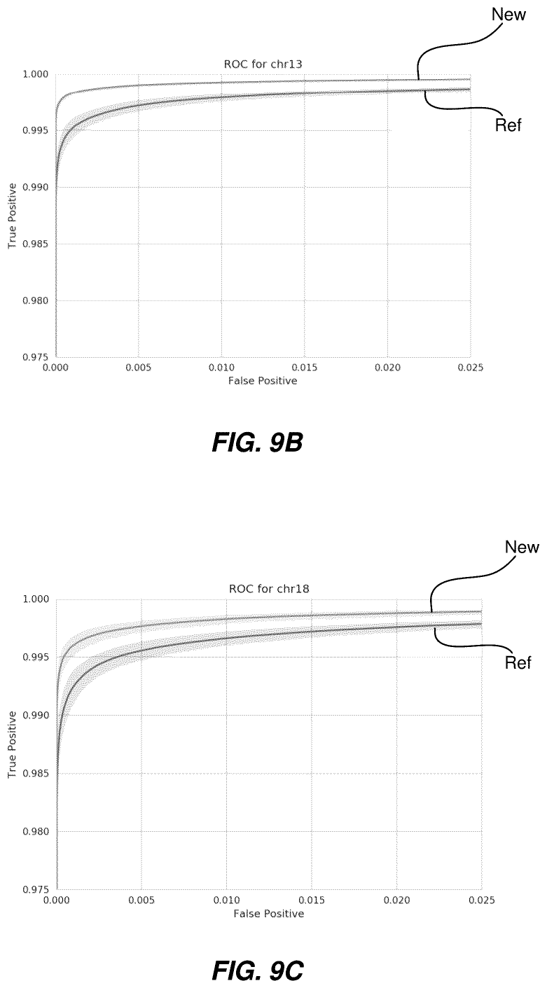

FIG. 9A shows ROC curves generated for classifications of fetal aneuploidy at chromosome 21 by each of the reference caller (“ref”) and the new candidate caller (“new”). The sensitivity (i.e., true-positive rate) was plotted versus 1−specificity (i.e., false-positive rate). As shown in FIG. 9A, the new caller performed significantly better than the reference caller, demonstrating increased sensitivity at each specificity. As shown, the false-positive rate may be reduced to approximately 0.3% while the true-positive rate is maintained above approximately 99.0%.

FIG. 9B shows ROC curves generated for classifications of fetal aneuploidy at chromosome 13 by each of the reference caller and the new candidate caller. As shown in FIG. 9B, the new caller performed significantly better than the reference caller, demonstrating increased sensitivity at each specificity.

FIG. 9C shows ROC curves generated for classifications of fetal aneuploidy at chromosome 18 by each of the reference caller and the new candidate caller. As shown in FIG. 9C, the new caller performed significantly better than the reference caller, demonstrating increased sensitivity at each specificity.

### Example 2: ROC Curve Construction and Comparison

ROC curves were generated in the manner described above in Example 1 to evaluate and compare two different aneuploidy callers utilized in noninvasive prenatal screening for aneuploidy detection. Performance of a reference caller was compared with performance of a new candidate caller having algorithmic enhancements over the reference caller.

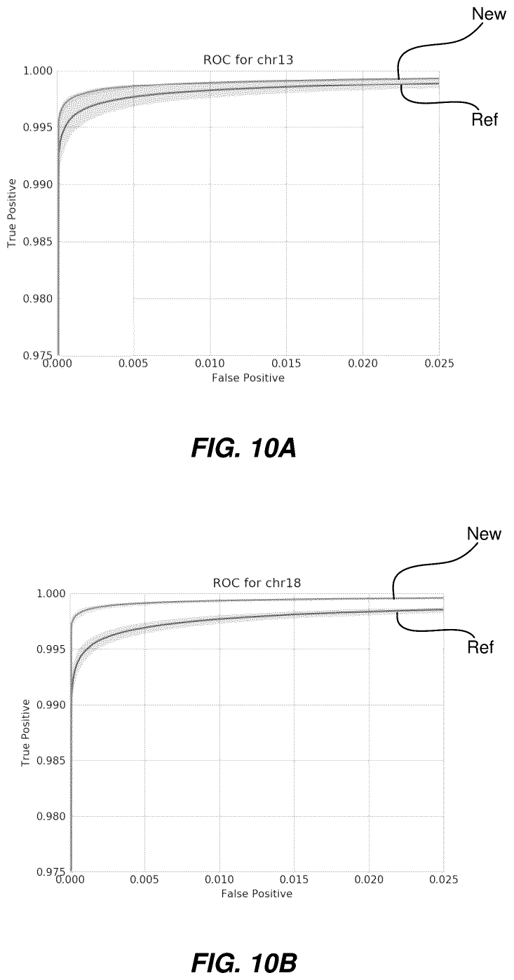

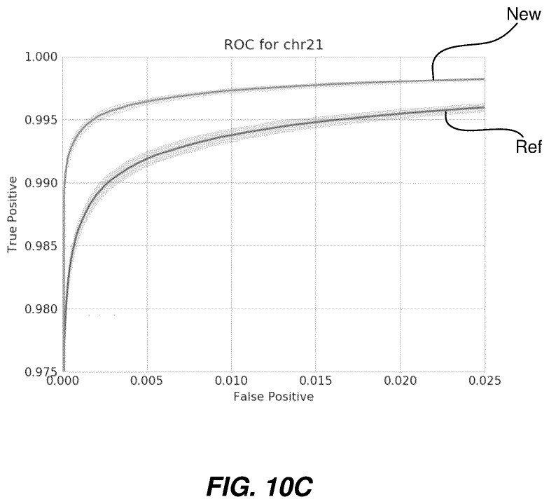

FIG. 10A shows ROC curves generated for classifications of fetal aneuploidy at chromosome 13 by each of the reference caller and the new candidate caller. As shown in FIG. 10A, the new caller performed significantly better than the reference caller, demonstrating increased sensitivity at each specificity.

FIG. 10B shows ROC curves generated for classifications of fetal aneuploidy at chromosome 18 by each of the reference caller and the new candidate caller. As shown in FIG. 10B, the new caller performed significantly better than the reference caller, demonstrating increased sensitivity at each specificity.

FIG. 10C shows ROC curves generated for classifications of fetal aneuploidy at chromosome 21 by each of the reference caller and the new candidate caller. As shown in FIG. 10C, the new caller performed significantly better than the reference caller, demonstrating increased sensitivity at each specificity.

### Example 3: Trajectories of Parameters During MCMC Sampling

Inference of the Bayesian model described in Example 1 on cohort data was performed for each of the reference caller dataset and the new caller dataset using MCMC sampling to explore the full posterior distributions for the model parameters of aneuploidy (trisomy) positive-call rate, z-score, and random variables m_{FF} (capturing the empirical relationship between z-score and fetal fraction), m_{depth} (capturing the empirical relationship between z-score and sequencing depth), and inverse variance τ_{z}. The MCMC sampling allows capture of the trajectories of the parameters over the course of sampling, as well as histograms of the data.

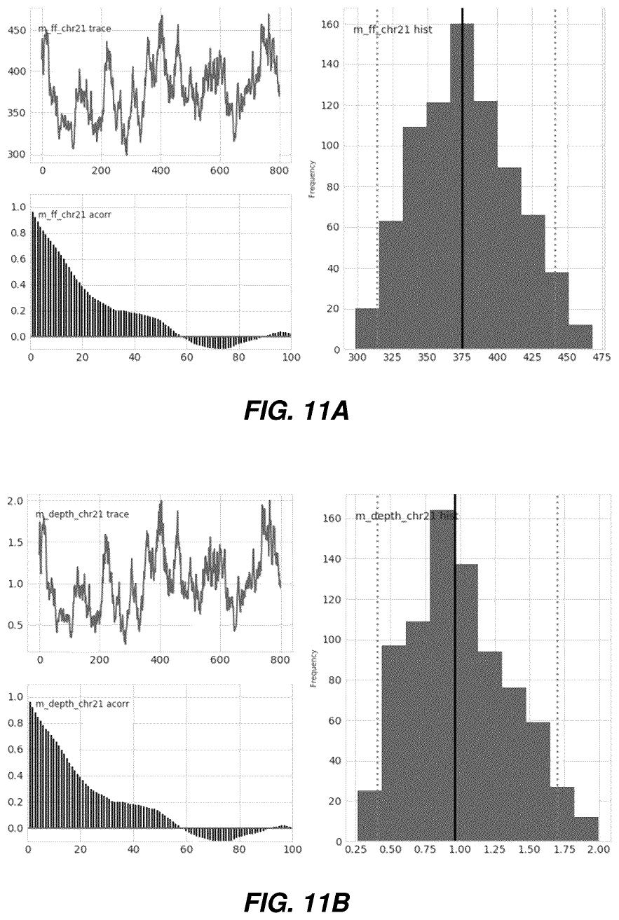

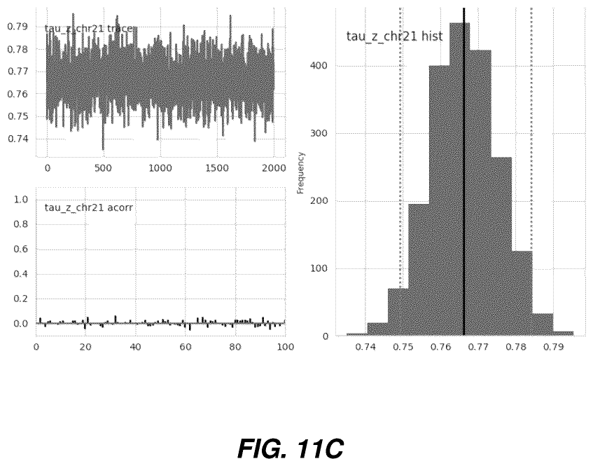

FIG. 11A shows the trajectory of m_{FF} over the course of MCMC sampling, as well as a histogram of the trisomy positive-call frequency relative to m_{FF}. FIG. 11B shows the trajectory of m_{depth} over the course of MCMC sampling, as well as a histogram of the trisomy positive-call frequency relative to m_{depth}. FIG. 11C shows the trajectory of inverse variance τ_{z} for z-score in chromosome 21 during MCMC sampling inference, as well as a histogram of the trisomy positive-call frequency relative to τ_{z}.

### Example 4: Posterior Predictive Z-Score Distributions

For the Bayesian model described in Example 1, MCMC predicted data by sampling fetal fraction from the trained prior, positive/negative labels from the trained positive-call rate, and finally z-scores given the learned dependence on fetal fraction and depth was determined. Using these posterior predictive samples, the estimated z-score distributions were reconstructed for false/true trisomy 21 for both the reference caller and the new candidate caller.

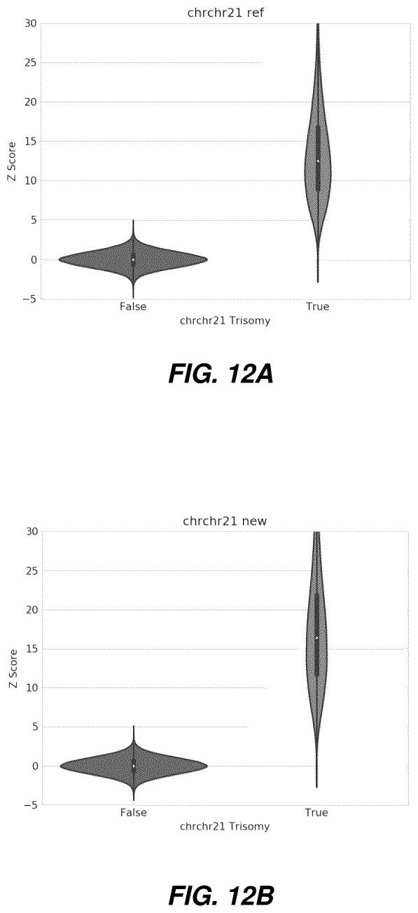

FIG. 12A shows the estimated z-score distributions reconstructed for false/true trisomy 21 for the reference caller based on 15,725 random anonymized clinical maternal samples. FIG. 12B shows the estimated z-score distributions reconstructed for false/true trisomy 21 for the new candidate caller based on the clinical maternal samples. As shown in these figures, the negative z-score distributions were essentially unchanged between the reference and new callers, while the positive z-score distributions were shifted to higher z-scores in the new caller, indicating improved classifier performance of the new caller.

### Example 5: Parameter Inference

For noninvasive prenatal screening, the z-score for a region assesses the normalized enrichment in mapped whole-genome sequencing reads. 15,725 assayed maternal samples were analyzed with both a reference caller (i.e., ref) and a new caller (i.e., new) algorithm pipeline. The vast majority of these maternal samples had no reported clinical outcomes, so true caller performance could not be directly obtained. Therefore, candidate z-score threshold limits of 3.0 and 3.4 for calling trisomy 21 were applied to the reference and new caller, respectively, to attain hypothetical concordance of called trisomy 21 between the two methods.

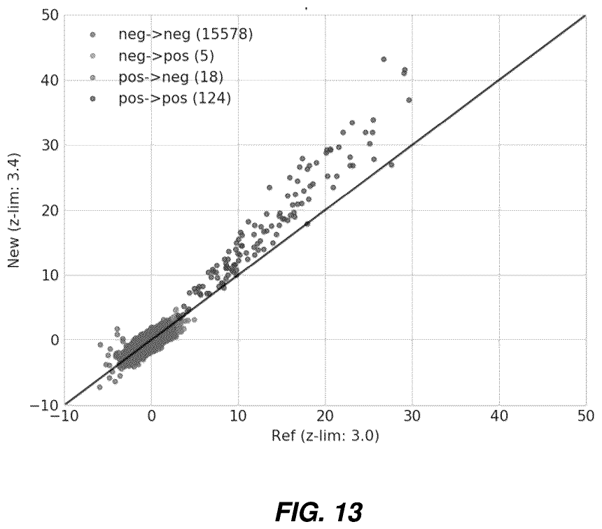

FIG. 13 shows z-score results of the new caller plotted against the reference caller. From the reference caller to the new caller, 5 maternal samples changed from a negative trisomy call to a positive trisomy call and 18 maternal samples changed from a positive trisomy call to a negative trisomy call, while 15,578 maternal samples were called as negative and 124 maternal samples were called as positive with both the reference caller and the new caller.

Using a reported clinical sensitivity 99.2% (98.5%-99.6%) and specificity 99.91% (99.86%-99.95%), as detailed in Gil et al., Fetal Diagnosis and Therapy, vol. 35(3), pp. 156-73 (2014), one would expect from a high risk population (i.e., with 1/125 incidence of trisomy 21) the following clinical trisomy outcomes shown in Table 5 for a similar hypothetical cohort of 15,725 maternal. This illustrates the magnitude of discordance from bioinformatics pipeline changes is commensurate and inherent to the statistical performance of the assay.

TABLE 5
Expected Clinical Trisomy Outcomes

| Chromosome 21 | True Positive | | False Positive | |
|---|---|---|---|---|
| Called Positive | 125 | (124-126) | 14 | (8-22) |
| Called Negative | 1 | (0.5-2) | 15,585 | (15,577-15,591) |

What is claimed is:

1. A computer-implemented method comprising:
sequencing a maternal sample of a pregnant patient to obtain at least 6 million sequencing reads, the maternal sample comprising cell-free DNA circulating in the maternal bloodstream, the cell-free DNA including fetal cell-free DNA and maternal cell-free DNA;
using a trained machine-learning model to measure a fetal fraction of the cell-free DNA in the maternal sample based at least on a count of binned sequencing reads from an interrogated region in the maternal sample, wherein using the trained machine-learning model comprises feeding a bin count vector to the trained machine-learning model and receiving the fetal fraction as an output, the machine-learning model trained by:
for each reference maternal sample in a plurality of reference maternal samples, measuring a dosage of a test chromosome in the reference maternal sample, and measuring a second dosage of a second chromosome other than the test chromosome in the reference maternal sample; and
applying a machine learning technique to a training data set comprising (i) the measured dosages for the test chromosome, and (ii) the second measured dosages for the second chromosome;
determining, using the fetal fraction, a first fetal chromosomal abnormality classification for the maternal sample using a first classifier and a second fetal chromosomal abnormality classification for the maternal sample using a second classifier;
generating ROC curves for each the first caller and the second caller using a Bayesian graphical modeling approach;
determining, based on the ROC curves, that the second caller has at least one of a higher sensitivity or a higher specificity than the first caller;
selecting the second fetal chromosomal abnormality classification of the second caller based at least on the higher sensitivity or the higher specificity of the second caller; and
reporting or displaying the second abnormality classification to at least one of the patient or a healthcare provider.

2. The method of claim 1, wherein each of the ROC curves represents a true-positive rate versus a false-positive rate of the corresponding caller in calling the fetal chromosomal abnormality in the test chromosome.

3. The method of claim 1, further comprising determining a value of statistical significance for each reference maternal sample based on a measured dosage, an expected dosage, and an expected variance in a number of sequencing reads per bin for the test chromosome of the reference maternal sample, wherein the value of statistical significance is a z-score, a p-value, or a probability.

4. The method of claim 1, further comprising determining a sequencing read depth for each of the plurality of reference maternal samples based on a number of de-duplicated mapped reads within an interrogated region from the reference maternal sample.

5. The method of claim 4, further comprising determining a value of statistical significance for each reference maternal sample based on a measured dosage, an expected dosage, and an expected variance in a number of sequencing reads per bin for the test chromosome of the reference maternal sample, and determining a relationship between the values of statistical significance and the sequencing read depths for the plurality of reference maternal samples.

6. The method of claim 1, further comprising determining a value of statistical significance for each reference maternal sample based on a measured dosage, an expected dosage, and an expected variance in a number of sequencing reads per bin for the test chromosome of the reference maternal sample, and determining a distribution of the values of statistical significance for the test chromosome of the plurality of reference maternal samples.

7. The method of claim 6, further comprising generating simulated maternal samples based on the relationship between the values of statistical significance and the abnormality classifications for the plurality of reference maternal samples, wherein the simulated maternal samples represent maternal samples having a different mean sequencing depth than the plurality of reference maternal samples, wherein determining the value of statistical significance predicted for the test chromosome of each of the plurality of simulated maternal samples comprises predicting the value of statistical significance for the test chromosome of each of the plurality of simulated maternal samples based on the distribution of values of statistical significance for the test chromosome of the plurality of reference maternal samples.

8. The method of claim 7, further comprising calculating an average number of sequencing reads per bin and an expected variance in the number of sequencing reads per bin for the test chromosome for each of the plurality of simulated maternal samples;
wherein predicting the value of statistical significance for the test chromosome of each of the plurality of simulated maternal samples further comprises predicting the value of statistical significance for the test chromosome of each of the plurality of simulated maternal samples based on the average number of sequencing reads per bin and the variance in the number of sequencing reads per bin for the test chromosome.

9. The method of claim 1, further comprising determining a value of statistical significance for each reference maternal sample based on a measured dosage, an expected dosage, and an expected variance in a number of sequencing reads per bin for the test chromosome of the reference maternal sample, and determining a relationship between the values of statistical significance and abnormality classifications for the plurality of reference maternal samples, wherein determining the relationship between the values of statistical significance and the abnormality classifications for the plurality of reference maternal samples comprises determining prior distributions of a plurality of inferred latent variables related to the values of statistical significance and the abnormality classifications for the test chromosome of the plurality of reference maternal samples.

10. The method of claim 9, wherein determining the prior distributions of the plurality of inferred latent variables related to the values of statistical significance and the abnormality classifications for the test chromosome of the plurality of reference maternal samples comprises performing Markov Chain Monte Carlo sampling using sequencing reads obtained from the plurality of reference maternal samples.

11. The method of claim 1, further comprising comparing a first ROC curve for the first caller to a second ROC curve for the second caller.

12. The method of claim 1, further comprising normalizing the number of sequencing reads and thereafter counting the sequencing reads.

13. The method of claim 1, wherein the fetal chromosomal abnormality is aneuploidy.

14. The method of claim 13, wherein the aneuploidy is monosomy or trisomy.

15. The method of claim 1, wherein the fetal chromosomal abnormality is a microdeletion.

16. The method of claim 1, wherein the test chromosome comprises chromosome 13, 18, 21, X, or Y.

17. The method of claim 1, wherein measuring the dosage of the test chromosome comprises:
obtaining reference sequence reads for the test chromosome;
aligning, by the computer processor, the reference sequence reads using a reference genome;
aggregating the reference sequence reads into reference bins;
determining, by the computer processor, a number of reference sequence reads in each reference bin; and
generating an average number of reference sequence reads and a variation of the number of reference sequence reads in the reference bins.

18. The method of claim 17, wherein measuring the second dosage of the second chromosome comprises:
obtaining second reference sequence reads for the second chromosome;
aligning the second reference sequence reads using the reference genome;
aggregating the second reference sequence reads into second reference bins;
determining a second number of reference sequence reads in each second reference bin; and
generating a second average number of second reference sequence reads and a second variation of the second number of second reference sequence reads in the second reference bins.

19. A system comprising:
A non-transitory machine-readable computer medium comprising instructions that when executed cause a processor to:
sequence a maternal sample of a pregnant patient to obtain at least 6 million sequencing reads, the maternal sample comprising cell-free DNA circulating in the maternal bloodstream, the cell-free DNA including fetal cell-free DNA and maternal cell-free DNA;
use a trained machine-learning model to measure a fetal fraction of the cell-free DNA in the maternal sample based at least on a count of binned sequencing reads from an interrogated region in the maternal sample, wherein using the trained machine-learning model comprises feeding a bin count vector to the trained machine-learning model and receiving the fetal fraction as an output, the machine-learning model trained by:
for each reference maternal sample in a plurality of reference maternal samples, measure a dosage of a test chromosome in the reference maternal sample, and measuring a second dosage of a second chromosome other than the test chromosome in the reference maternal sample;
apply a machine learning technique to a training data set comprising (i) the measured dosages for the test chromosome, and (ii) the second measured dosages for the second chromosome;
determine, using the fetal fraction, a first fetal chromosomal abnormality classification for the maternal sample using a first classifier and a second fetal chromosomal abnormality classification for the maternal sample using a second classifier;
generate ROC curves for each the first caller and the second caller using a Bayesian graphical modeling approach;
determine, based on the ROC curves, that the second caller has at least one of a higher sensitivity or a higher specificity than the first caller;
select the second fetal chromosomal abnormality classification of the second caller based at least on the higher sensitivity or the higher specificity of the second caller; and
report or displaying the second abnormality classification to at least one of the patient or a healthcare provider.

20. The system of claim 19, wherein each of the ROC curves represents a true-positive rate versus a false-positive rate of the corresponding caller in calling the fetal chromosomal abnormality in the test chromosome.
